(19) United States
(12) Patent Application Publication
(10) Pub. No.: US 2019/0300867 A1
(43) Pub. Date: Oct. 3, 2019

(54) BYPASSING THE PAM REQUIREMENT OF THE CRISPR-CAS SYSTEM

(71) Applicant: The Trustees of Columbia University in the City of New York, New York, NY (US)

(72) Inventor: Suleyman UCUNCUOGLU, New York, NY (US)

(21) Appl. No.: 16/302,833

(22) PCT Filed: May 22, 2017
(86) PCT No.: PCT/US17/33835

§ 371 Date: Nov. 19, 2018

Related U.S. Application Data
(60) Provisional application No. 62/340,265, filed on May 23, 2016.
(60) Provisional application No. 62/479,109, filed on Mar. 30, 2017.

(51) Int. Cl. C12N 9/22; C12N 15/11

(52) U.S. Cl. CPC C12N 9/22; C12N 2310/20; C12N 15/11

(57) ABSTRACT

The present CRISPR-Cas9 systems can cleave a double-stranded DNA (dsDNA) independent of the protospacer adjacent motif (PAM). By utilizing an invader RNA (iRNA) to separate at least one portion of the dsDNA, the present system and method offer great flexibility to modify a large range of DNA targets.

### CROSS REFERENCE TO RELATED APPLICATIONS

This application claims priority to U.S. Provisional Application Nos. 62/340,265 (filed on May 23, 2016) and 62/479,109 (filed on Mar. 30, 2017), which are incorporated herein by reference in their entirety.

### GOVERNMENT LICENSE RIGHTS

This invention was made with government support under Grant No. 1DP2EB018657-01 awarded by the National Institutes of Health (NIH). The government may have certain rights in the invention.

### SEQUENCE LISTING

The instant application contains a Sequence Listing which has been filed electronically in ASCII format and is hereby incorporated by reference in its entirety. Said ASCII copy, created on May 22, 2017, is named 01001-004999-WO0_SL.txt and is 6 KB in size.

### FIELD OF THE INVENTION

The present invention relates to methods and systems for modifying DNA and for gene targeting. In particular, the present invention relates to utilizing the Clustered Regularly Interspaced Short Palindromic Repeats (CRISPR)-Cas systems to target (e.g., cleave) a double-stranded DNA (dsDNA) independent of the protospacer adjacent motif (PAM).

### BACKGROUND OF THE INVENTION

The Cas/CRISPR system is a prokaryotic immune system that confers resistance to foreign genetic elements such as plasmids and bacteriophages. The CRISPR/Cas9 system exploits RNA-guided DNA-binding and sequence-specific cleavage of a target DNA. A guide RNA (gRNA) are complementary to a target DNA sequence upstream of a PAM (protospacer adjacent motif) site. The Cas (CRISPR-associated) 9 protein binds to the gRNA and the target DNA and introduces a double-strand break (DSB) in a defined location upstream of the PAM site. Geurts et al., Science 325, 433 (2009); Mashimo et al., PLoS ONE 5, e8870 (2010); Carbery et al., Genetics 186, 451-459 (2010); Tesson et al., Nat. Biotech. 29, 695-696 (2011). Wiedenheft et al. Nature 482,331-338 (2012); Jinek et al. Science 337,816-821 (2012); Mali et al. Science 339,823-826 (2013); Cong et al. Science 339,819-823 (2013). The ability of the CRISPR/Cas9 system to be programed to cleave not only viral DNA but also other genes opened a new venue for genome engineering.

PAM is a DNA sequence immediately following the DNA sequence targeted by the CRISPR/Cas9 system. It has been reported that PAM plays an essential role in DNA target recognition. Doudna et al., The new frontier of genome engineering with CRISPR/Cas9, Science, 2014, 346(6213): 1258096. Although CRISPR/Cas9 nucleases are widely used for genuine editing, the range of sequences that Cas9 can recognize is constrained by the need for a specific PAM. As a result, it can often be difficult to introduce double-strand breaks (DSBs) with the precision that is necessary for various genome-editing applications. For example, CRISPR/Cas9 system can be used to either repress or edit genes. To repress effectively, the target needs to be close to the promoter sequence. However, the target gene may not have a PAM region, or the PAM region may be at an undesirable location. Furthermore, the PAM requirement also increases the likelihood of off-target mutations on other chromosomes. Kuscu et al., Genome-wide analysis reveals characteristics of off-target sites bound by the Cas9 endonuclease, Nature biotechnology 32. No, 7 (2014): 677-683.

For the Cas9 nuclease of Streptococcus pyogenes, the canonical PAM is the sequence 5′-NGG-3′ where “N” is any nucleotide. Different PAMs are associated with the Cas9 proteins of other bacteria such as Neisseria meningitidis, Treponema denticola, and Streptococus thermophilus. For example, non-canonical PAM may be the sequence 5′-NGA-3′ or 5′-NAG-3′.

Attempts have been made to engineer Cas9 enzymes to provide Cas9 variants with altered PAM specificities. Kleinstiver et al., Engineered CRISPR-Cas9 nucleases with altered PAM specificities, Nature, 2015, 523: 481-485.

Recently, Ma et al. reported that CRISPR-Cas9 system can cleave single-stranded DNA independent of the PAM region. Ma et al., Single-Stranded DNA Cleavage by Divergent CRISPR-Cas9 Enzymes, Mol. Cell, 2015, 60 (3), p398-407.

The present application provides for CRISPR-Cas9 systems that can cleave double-stranded DNA (dsDNA) targets independent of the PAM region. Thus, it becomes possible to target any DNA sequences even if there is no PAM region. This invention provides flexibility to choose any target sequence with the CRISPR/Cas9 system.

### SUMMARY

The present disclosure provides for a system that targets a target sequence in a double-stranded DNA, the system comprising: (i) a first RNA (e.g., sgRNA) comprising: (a) a first segment (e.g., crRNA or gRNA) that hybridizes with the target sequence in a target strand of the double-stranded DNA; and (b) a second segment (e.g., tracrRNA) that hybridizes with the first segment to form a double-stranded protein-binding motif; (ii) a second RNA (e.g., iRNA) that hybridizes with a sequence in a non-target strand of the double-stranded DNA; and (iii) a Cas enzyme or a variant thereof, wherein the first RNA (e.g., sgRNA) forms a complex with the Cas enzyme or a variant thereof.

In certain embodiments, the second RNA has about 14 to about 34 nucleotides.

The present disclosure provides for a DNA-targeting RNA (e.g., isgRNA), comprising: (i) a first segment (e.g., crRNA or gRNA) that hybridizes with a target sequence in a target strand of a double-stranded DNA; (ii) a second segment (e.g., tracrRNA) that hybridizes with the first segment to form a double-stranded protein-binding motif; and (iii) a third segment (e.g., iRNA) that hybridizes with a sequence in a non-target strand of the double-stranded DNA, wherein the RNA forms a complex with a Cas enzyme or a variant thereof.

In certain embodiments, the third segment has about 14 to about 34 nucleotides.

The present disclosure provides for a system that targets a target sequence in a double-stranded DNA, the system comprising: (i) a first RNA (e.g., crRNA or gRNA) that hybridizes with the target sequence in a target strand of the double-stranded DNA; and (ii) a second RNA (e.g., tracrRNA) that hybridizes with the first RNA to form a double-stranded protein-binding motif; (iii) a third RNA (e.g., iRNA) that hybridizes with a sequence in a non-target strand of the double-stranded DNA; and (iv) a Cas enzyme or a variant thereof.

In certain embodiments, the third RNA has about 14 to about 34 nucleotides.

The present disclosure provides for a system that targets a target sequence in a double-stranded DNA, the system comprising: (i) a first DNA polynucleotide encoding a first RNA (e.g., sgRNA), the first RNA comprising: (a) a first segment (e.g., crRNA or gRNA) that hybridizes with a target sequence in a target strand of the double-stranded DNA; and (b) a second segment (e.g., tracrRNA) that hybridizes with the first segment to form a double-stranded protein-binding motif; (ii) a second DNA polynucleotide encoding a second RNA (e.g., iRNA), wherein the second RNA hybridizes with a sequence in a non-target strand of the double-stranded DNA; and (iii) a third DNA polynucleotide encoding a Cas enzyme or a variant thereof.

In certain embodiments, the second RNA has about 14 to about 34 nucleotides.

In certain embodiments, the first DNA polynucleotide, the second DNA polynucleotide, and the third DNA polynucleotide are within a vector (located on the same vector). In certain embodiments, the first DNA polynucleotide, the second DNA polynucleotide, and the third DNA polynucleotide are located on different vectors (e.g., two, three or more vectors).

The present disclosure provides for a system that targets a target sequence in a double-stranded DNA, the system comprising: (i) a first DNA polynucleotide encoding a first RNA (e.g., isgRNA), the first RNA comprising: (a) a first segment (e.g., crRNA or gRNA) that hybridizes with a target sequence in a target strand of the double-stranded DNA; and (b) a second segment (e.g., tracrRNA) that hybridizes with the first segment to form a double-stranded protein-binding motif; and (c) a third segment (e.g., iRNA) that hybridizes with a sequence in a non-target strand of the double-stranded DNA; (ii) a second DNA polynucleotide encoding a Cas enzyme or a variant thereof.

In certain embodiments, the third segment has about 14 to about 34 nucleotides.

In certain embodiments, the first DNA polynucleotide and the second DNA polynucleotide are within a vector (located on the same vector). In certain embodiments, the first DNA polynucleotide and the second DNA polynucleotide are located on different vectors (e.g., two or more vectors).

The present disclosure provides for a system that targets a target sequence in a double-stranded DNA, the system comprising: (i) a first DNA polynucleotide encoding a first RNA (e.g., crRNA or gRNA), wherein the first RNA hybridizes with a target sequence in a target strand of a double-stranded DNA; (ii) a second DNA polynucleotide encoding a second RNA (e.g., tracrRNA), wherein the second RNA hybridizes with the first RNA to form a double-stranded protein-binding motif; (iii) a third DNA polynucleotide encoding a third RNA (e.g., iRNA), wherein the third RNA hybridizes with a sequence in a non-target strand of the double-stranded DNA; and (iv) a fourth DNA polynucleotide encoding a Cas enzyme or a variant thereof.

In certain embodiments, the third RNA has about 14 to about 34 nucleotides.

In certain embodiments, the first DNA polynucleotide, the second DNA polynucleotide, the third DNA polynucleotide, and the fourth DNA polynucleotide are within a vector (located on the same vector). In certain embodiments, the first DNA polynucleotide, the second DNA polynucleotide, the third DNA polynucleotide, and the fourth DNA polynucleotide are located on different vectors (e.g., two, three, four or more vectors).

The present disclosure provides for a method of targeting a target sequence in a double-stranded DNA, the method comprising the step of contacting the double-stranded DNA with a system comprising: (i) a first RNA, or a DNA polynucleotide encoding a first RNA, wherein the first RNA (e.g., sgRNA) comprises: (a) a first segment (e.g., crRNA or gRNA) that hybridizes with a target sequence in a target strand of the double-stranded DNA; (b) a second segment (e.g., tracrRNA) that hybridizes with the first segment to form a double-stranded protein-binding motif; (ii) a second RNA, or a DNA polynucleotide encoding a second RNA, wherein the second RNA (e.g., iRNA) hybridizes with a sequence in a non-target strand of the double-stranded DNA; and (iii) a Cas enzyme protein or a variant thereof, or a DNA polynucleotide or a RNA polynucleotide encoding a Cas enzyme or a variant thereof.

In certain embodiments, the second RNA has about 14 to about 34 nucleotides.

The present disclosure provides for a method of targeting a target sequence in a double-stranded DNA, the method comprising the step of contacting the double-stranded DNA with a system comprising: (i) a DNA-targeting RNA, or a DNA polynucleotide encoding a DNA-targeting RNA, wherein the DNA-targeting RNA (e.g., isgRNA) comprises: (a) a first segment (e.g., crRNA or gRNA) that hybridizes with a target sequence in a target strand of the double-stranded DNA; (b) a second segment (e.g., tracrRNA) that hybridizes with the first segment to form a double-stranded protein-binding motif; and (c) a third segment (e.g., iRNA) that hybridizes with a sequence in a non-target strand of the double-stranded DNA; and (ii) a Cas enzyme or a variant thereof, or a DNA polynucleotide or a RNA polynucleotide encoding a Cas enzyme or a variant thereof.

The present disclosure provides for a method of targeting a target sequence in a double-stranded DNA in a cell, the method comprising the step of introducing into the cell a system comprising: (i) a first RNA, or a DNA polynucleotide encoding a first RNA, wherein the first RNA (e.g., sgRNA) comprises: (a) a first segment (e.g., crRNA or gRNA) that hybridizes with a target sequence in a target strand of the double-stranded DNA; and (b) a second segment (e.g., tracrRNA) that hybridizes with the first segment to form a double-stranded protein-binding motif; and (ii) a second RNA, or a DNA polynucleotide encoding a second RNA, wherein the second RNA (e.g., iRNA) hybridizes with a sequence in a non-target strand of the double-stranded DNA.

In certain embodiments, the cell expresses a Cas enzyme.

In certain embodiments, the method further comprises delivering into the cell (i) a DNA polynucleotide encoding a Cas enzyme or a variant thereof, (ii) a RNA polynucleotide encoding a Cas enzyme or a variant thereof, or (iii) a Cas enzyme or a variant thereof.

The present disclosure provides for a method of targeting a target sequence in a double-stranded DNA in a cell, the method comprising the step of introducing into the cell a DNA-targeting RNA, or a DNA polynucleotide encoding a DNA-targeting RNA, wherein the DNA-targeting RNA (e.g., isgRNA) comprises: (a) a first segment (e.g., crRNA or gRNA) that hybridizes with a target sequence in a target strand of the double-stranded DNA; (b) a second segment (e.g., tracrRNA) that hybridizes with the first segment to form a double-stranded protein-binding motif; and (c) a third segment (e.g., iRNA) that hybridizes with a sequence in a non-target strand of the double-stranded DNA.

In certain embodiments, the cell expresses a Cas enzyme.

In certain embodiments, the method further comprises delivering into the cell (i) a DNA polynucleotide encoding a Cas enzyme or a variant thereof, (ii) a RNA polynucleotide encoding a Cas enzyme or a variant thereof, or (iii) a Cas enzyme or a variant thereof.

The target sequence may or may not be immediately flanked by a protospacer adjacent motif (PAM).

The present disclosure also provides for a polynucleotide, such as a DNA polynucleotide, encoding one or more of the present CRISPR components including the iRNA, isgRNA, sgRNA, crRNA, gRNA, tracrRNA etc., and the Cas enzyme or a variant thereof.

The present disclosure provides for a vector (or a construct, etc.) comprising the present polynucleotide, such as the present DNA polynucleotide. The vector (or construct, etc.) encodes one or more of the present CRISPR components including the iRNA, isgRNA, sgRNA, crRNA, gRNA, tracrRNA etc., and the Cas enzyme or a variant thereof.

The present disclosure provides for a cell comprising the present polynucleotide, such as the present DNA polynucleotide, the present vector (or construct, etc.), and/or one or more of the present CRISPR components including the iRNA, isgRNA, sgRNA, crRNA, gRNA, tracrRNA etc., and the Cas enzyme or a variant thereof.

The present disclosure provides for a kit comprising the present system, the present polynucleotide, such as the present DNA polynucleotide, the present vector (or construct, etc.), and/or one or more of the present CRISPR components including the iRNA, isgRNA, sgRNA, crRNA, gRNA, tracrRNA etc., and the Cas enzyme or a variant thereof.

In certain embodiments, the Cas enzyme is Cas9, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, orthologs thereof, or modified versions thereof. In one embodiment, the Cas enzyme is Cas9.

In certain embodiments, the Cas enzyme comprises one or more mutations.

In certain embodiments, the Cas enzyme is codon-optimized for expression in a eukaryotic cell, such as a mammalian cell, or a human cell.

In certain embodiments, the Cas enzyme or a variant thereof cleaves the target sequence.

### BRIEF DESCRIPTION OF THE DRAWINGS

FIGS. 1A and 1B show different strategies to target a dsDNA with the CRISPR-Cas9 system in a PAM-independent manner. The invader RNA which hybridizes with the non-target DNA strand can be covalently linked to the sgRNA (FIG. 1A), or can be supplied separately in combination with the sgRNA (FIG. 1B).

FIG. 2 shows that a dsDNA can be cleaved in the absence of PAM by two different approaches involving an invader RNA. A 2148-bp dsDNA was targeted by either (i) Cas9 plus an isgRNA, or (ii) Cas9 plus an sgRNA and iRNA. The target regions of the dsDNA do not contain canonical PAM sequences. The reactions were loaded to a 1% agarose gel and analyzed. The two isgRNAs were named as isgRNA 580 and isgRNA 591 based on their respective cleavage sites in the dsDNA. Similarly, the two sgRNAs were named as sgRNA 580 and sgRNA 591 based on their respective cleavage sites in the dsDNA. Lane 3: isgRNA 591 (sgRNA covalently linked to invader RNA) +Cas9; molar ratios of DNA:Cas9:isgRNA 591 are 1:10:10. Cleaved DNA can be seen. Lane 4: isgRNA 580 (sgRNA covalently linked to invader RNA) +Cas9; molar ratios of DNA:Cas9:isgRNA 580 are 1:10:10. Cleaved DNA can be seen. Lanes 6-10: Cas9+sgRNA (crRNA-tracrRNA)+a separate invader RNA. Lane 6: molar ratios of DNA:Cas9:sgRNA 591:iRNA 591 are 1:10:10:100. Lane 7: molar ratios of DNA:Cas9:sgRNA 580:iRNA 580 are 1:10:10:100. Lane 8: molar ratios of DNA:Cas9:sgRNA 591:iRNA 591 are 1:10:10:1000. Lane 9: molar ratios of DNA:Cas9:sgRNA 580:iRNA 580 are 1:10:10:1000. Lane 10: molar ratios of DNA:Cas9:sgRNA iRNA 580:Cas9:sgRNA 591:iRNA 591 are 1 DNA 10:Cas9:10 sgRNA 580:100 iRNA 580:10 Cas9:10 sgRNA 591:100 iRNA 591. For the reaction of lane 10, the set of Cas9, sgRNA 591 and iRNA 591 was added 30 minutes later than the set of Cas9, sgRNA 580 and iRNA 580. Control assays include the following: lane 1: dsDNA only; lane 2: Cas9 without any guide RNA; lane 5: Cas9 with a wild type sgRNA (no invader RNA). For lane 5, the molar ratios of DNA:Cas9:sgRNA are 1:10:10. Lane M: 1 kb DNA ladder (NEB).

FIG. 3 shows that Cas9 cleaves a dsDNA substrate in the absence of PAM by two different approaches involving an invader RNA. A 2148-bp dsDNA was targeted by either (i) Cas9 plus an isgRNA, or (ii) Cas9 plus an sgRNA and iRNA. The target regions of the dsDNA do not contain canonical PAM sequences. The reactions were loaded to a 1% agarose gel and analyzed. The two isgRNAs were named as isgRNA 580 and isgRNA 591 based on their respective cleavage sites in the dsDNA. The two sgRNAs were named as sgRNA 580 and sgRNA 591 based on their respective cleavage sites in the dsDNA. Lane 1: 2 kb-dsDNA only (control). Lane 2: 2 kb-dsDNA (with no PAM near target sequence)+isgRNA 580, Lane 3: 2 kb-dsDNA (with no PAM near target sequence)+isgRNA 591. Lane 4: 2 kb-dsDNA (with no PAM near target sequence)+sgRNA 580+invader RNA 580. Lane 5: 2 kb-dsDNA (with no PAM near target sequence)+sgRNA 591+invader RNA 591. Lane 6: 2 kb-dsDNA (with no PAM near target sequence)+sgRNA 580+sgRNA 591+corresponding invader RNAs (iRNA 580 and iRNA 591). Cas9 was also added to lanes 2-6.

FIG. 4 shows that Cas9 cleaves an ssDNA substrate in the absence of PAM by two different approaches involving an invader RNA. Four different assays were prepared with two 80-nt ssDNA substrates. The assays had (i) the ssDNA only (lanes 2 and 3); (ii) the ssDNA and Cas9 without any guide RNA (lanes 4 and 5); (iii) the ssDNA, Cas9, and sgRNA (crRNA-tracrRNA) and a 28-nt iRNA which hybridizes with a sequence of the ssDNA (lanes 6 and 8); or (iv) ssDNA and iRNA (lanes 7 and 9). The reactions were loaded to 4% agarose gel which was stained with Gelred (Biotium). Lane 1: O'RangeRuler 10 bp DNA Ladder, Lane 2: ssDNA-1 only. Lane 3: ssDNA-2 only. Lane 4: ssDNA-2+4. Cas9, Lane 5: ssDNA-1+Cas9. Lane 6: ssDNA-1+Cas9+crRNA-tracrRNA+iRNA. Lane 7: ssDNA-1+iRNA. Lane 8: ssDNA-2+Cas9+crRNA-tracrRNA+iRNA. Lane 9: ssDNA-2+iRNA

### DETAILED DESCRIPTION

The present disclosure provides for CRISPR-Cas systems that can target (e.g., cleave) a double-stranded DNA (dsDNA) independent of the PAM region. By utilizing an invader RNA (iRNA) to separate at least one portion of the dsDNA, the present system and method offer great flexibility to modify a large range of DNA targets.

In certain embodiments, a portion of the target dsDNA is transiently separated by an invader RNA which hinds to the non-target strand of the dsDNA. The transition from dsDNA to two ssDNA strands creates a bulged dsDNA. Following bulging, a guide RNA binds to the target sequence of the target strand of the dsDNA, which will allow the Cas enzyme to target (e.g., cleave) the ssDNA without the need for a PAM region. In one embodiment, strand separation of the dsDNA and Cas binding are synchronized.

Once the first strand of the target DNA is targeted (e.g., cleaved), the second strand may or may not be targeted similarly using the present CRISPR-Cas system independent of the PAM region. In certain embodiments, the second strand of the target DNA is targeted (e.g., cleaved) about 20 nucleotides, about 19 nucleotides, about 18 nucleotides, about 17 nucleotides, about 16 nucleotides, about 15 nucleotides, about 14 nucleotides, about 13 nucleotides, about 12 nucleotides, or about 11 nucleotides, from the target site (e.g., the cleavage site) of the first strand (upstream or downstream). In certain embodiments, the second strand of the target DNA is targeted (e.g., cleaved) more than about 20 nucleotides from the target site (e.g., the cleavage site) of the first strand (upstream or downstream). When both strands of the target DNA are cleaved by the present system, the double-strand break may produce a sticky-ended DNA or a blunt-ended DNA.

As used herein, an invader RNA (iRNA) is complementary to a nucleic acid sequence in a non-target strand of a dsDNA in vitro or in a host cell, whereas the gRNA targets the CRISPR/Cas complex to a target nucleic acid sequence in a target strand of a dsDNA. In general, an iRNA is any polynucleotide sequence having sufficient complementarity with a sequence in a non-target strand of a dsDNA to hybridize with the sequence, thus inducing separation of at least one portion of the dsDNA into ssDNAs.

Each construct or vector of the present system may encode or contain one, two or more iRNAs or isgRNAs. Multiple (two or more) iRNAs or isgRNAs can be used to assist the CRISPR-Cas system to target multiple different genes simultaneously, or target different sites of the same gene.

As used herein, a CRISPR component refers to any of, an iRNA, an isgRNA, a gRNA, a crRNA, a tracrRNA, an sgRNA, a chimeric RNA, and a Cas enzyme.

“Complementarity” refers to the ability of a nucleic acid to form hydrogen bond(s) with another nucleic acid sequence by either traditional Watson-Crick base pairing or other non-traditional types of pairing. “Substantially complementary” refers to a degree of complementarity that is about or more than about 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides (e.g., contiguous nucleotides), or refers to two nucleic acids that hybridize under stringent conditions. As used herein, “stringent conditions” for hybridization refers to conditions under which a nucleic acid having complementarity to a target sequence predominantly hybridizes with the target sequence, and substantially does not hybridize to non-target sequences. Stringent conditions are generally sequence-dependent, and vary depending on a number of factors. Non-limiting examples of stringent conditions are described in detail in Tijssen (1993), Laboratory Techniques in Biochemistry and Molecular Biology-Hybridization with Nucleic Acid Probes, Part I, Second Chapter “Overview of principles of hybridization and the strategy of nucleic acid probe assay”, Elsevier, N.Y.

The present disclosure provides for a DNA-targeting RNA, or DNA polynucleotide encoding a DNA-targeting RNA, where the DNA-targeting RNA comprises: (i) a first segment (e.g., crRNA or gRNA) that hybridizes with a target sequence in a target strand of a double-stranded DNA; (ii) a second segment (e.g., tracrRNA) that hybridizes with the first segment (e.g., to form a double-stranded protein-binding motif); and (iii) a third segment (e.g., invader RNA) that hybridizes with a sequence in a non-target strand of the double-stranded DNA. The RNA can form a complex with a Cas enzyme or a variant thereof.

The RNA that includes a first segment (e.g., crRNA or gRNA), a second segment (e.g., tracrRNA) and a third segment (e.g., invader RNA) may be referred to as “invader sgRNA” or “isgRNA”.

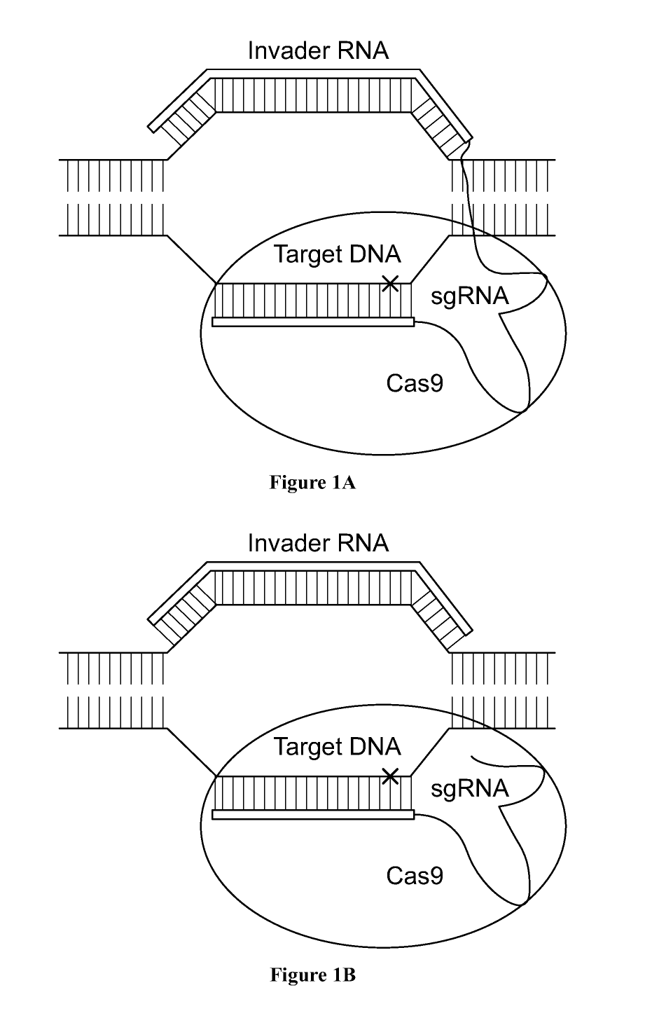

One embodiment of the present disclosure is shown in FIG. 1A.

The present disclosure also provides for a DNA-targeting system that comprises or encodes at least two RNA molecules: (i) a first RNA (e.g., an sgRNA comprising crRNA-tracrRNA) comprising: (a) a first segment (e.g., crRNA or gRNA) that hybridizes with the target sequence in a target strand of the double-stranded DNA; and (b) a second segment (e.g., tracrRNA) that hybridizes with the first segment to form a double-stranded protein-binding motif; and (ii) a second RNA (e.g., invader RNA) that hybridizes with a sequence in a non-target strand of the double-stranded DNA. The double-stranded protein-binding motif formed by the first segment and second segment of the first RNA may form a complex with a Cas enzyme or a variant thereof.

One embodiment of the present disclosure is shown in FIG. 1B.

The present disclosure provides for a system that targets a target sequence in a double-stranded DNA. The system may comprise or encode: (i) a first RNA (e.g., crRNA or gRNA) that hybridizes with the target sequence in a target strand utile double-stranded DNA; (ii) a second RNA (e.g., tracrRNA) that hybridizes with the first RNA to form a double-stranded protein-binding motif; (iii) a third RNA (e.g., invader RNA) that hybridizes with a sequence in a non-target strand of the double-stranded DNA; and (iv) a Cas enzyme or a variant thereof. The double-stranded protein-binding motif formed by the first RNA and the second RNA may form a complex with a Cas enzyme or a variant thereof.

The present disclosure provides for a DNA-targeting system that comprises or encodes at least two RNA molecules: (i) a first RNA comprising: (a) a first segment (e.g., crRNA or gRNA) that hybridizes with the target sequence in a target strand of the double-stranded DNA; and (b) a second segment (e.g., invader RNA) that hybridizes with a sequence in a non-target strand of the double-stranded DNA; and (ii) a second RNA (e.g., tracrRNA) that hybridizes with the first segment of the first RNA to form a double-stranded protein-binding motif. The double-stranded protein-binding motif may form a complex with a Cas enzyme or a variant thereof.

The present disclosure provides for a DNA-targeting system that comprises or encodes at least two RNA molecules: (i) a first RNA (e.g., crRNA or gRNA) that hybridizes with the target sequence in a target strand of the double-stranded DNA; and (ii) a second RNA comprising: (a) a first segment (e.g., tracrRNA) that hybridizes with the first RNA to form a double-stranded protein-binding motif; and (b) a second segment (e.g., invader RNA) that hybridizes with a sequence in a non-target strand of the double-stranded DNA. The double-stranded protein-binding motif may form a complex with a Cas enzyme or a variant thereof.

The present invader RNA may be replaced by, and/or be used in combination with, an invader polynucleotide, such as an invader ssDNA. The invader polynucleotide binds to the non-target strand of a dsDNA to separate at least one portion of the dsDNA.

The terms “polynucleotide”, “nucleotide”, “nucleotide sequence”, “nucleic acid” and “oligonucleotide” are used interchangeably. These terms refer to a polymeric form of nucleotides of any length, deoxyribonucleotides and/or ribonucleotides, or analogs thereof. An invader polynucleotide may be a DNA, an RNA, a DNA-RNA hybrid, a protein nucleic acid (PNA) formed by conjugating bases to an amino acid backbone, etc. An invader polynucleotide may also include a nucleic acid containing modified bases, for example, thio-uracil, thio-guanine and fluoro-uracil, etc. Invader polynucleotides encompass nucleic acids containing one or more nucleotide analogs or modified backbone residues or linkages, which are synthetic, naturally occurring, and non-naturally occurring. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, and carbamates) and with charged linkages (e.g., phosphorothioates, and phosphorodithioates). Polynucleotides may contain one or more additional covalently linked moieties, such as, for example, proteins (e.g., nucleases, toxins, antibodies, signal peptides, and poly-L-lysine), intercalators (e.g., acridine, and psoralen), chelators (e.g., metals, radioactive metals, iron, and oxidative metals), and alkylators. The polynucleotides may be derivatized by formation of a methyl or ethyl phosphotriester or an alkyl phosphoramidate linkage. Modifications of the ribose-phosphate backbone may be done to facilitate the addition of labels, or to increase the stability and half-life of such molecules in physiological environments. Nucleic acid analogs can find use in the methods of the invention as well as mixtures of naturally occurring nucleic acids and analogs. Furthermore, the polynucleotides herein may also be modified with a label capable of providing a detectable signal, either directly or indirectly. Exemplary labels include radioisotopes, fluorescent molecules, and biotin. In certain embodiments, one or more nucleotides within a polynucleotide are modified. In certain embodiments, the sequence of polynucleotide is interrupted by non-nucleotide components. In certain embodiments, a polynucleotide may also be modified after polymerization, such as by conjugation with a labeling agent.

The target sequence may or may not be immediately flanked by a protospacer adjacent motif (PAM).

In certain embodiments, the molar ratio of an iRNA to an sgRNA may range from about 500:1 to about 1:100, 400:1 to about 1:80, 300:1 to about 1:60, 200:1 to about 1:50, 200:1 to about 1:30, from about 200:1 to about 1:20, from about 150:1 to about. 1:15, from about 100:1 to about 1:10, from about 80:1 to about 1:5, from about 60:1 to about 1:2, from about 50:1 to about 1:1, from about 40:1 to about 1:1, from about 30:1 to about 1:1, from about 20:1 to about 1:1, from about 15:1 to about 1:1, from about 10:1 to about 1:1, from about 8:1 to about from about 6:1 to about 1:1, from about 5:1 to about 1:1, from about 4:1 to about 1:1, from about 3:1 to about 1:1, or from about 2:1 to about 1:1. In certain embodiments, the molar ratio of an iRNA to an sgRNA is about 10:1.

In certain embodiments, the molar ratio of an iRNA to a crRNA may range from about 500:1 to about 1:100, 400:1 to about 1:80, 300:1 to about 1:60, 200:1 to about 1:50, 200:1 to about 1:30, from about 200:1 to about 1:20, from about 150:1 to about 1:15, from about 100:1 to about 1:10, from about 80:1 to about 1:5, from about 60:1 to about 1:2, from about 50:1 to about 1:1, from about 40:1 to about 1:1, from about 30:1 to about 1:1, from about 20:1 to about 1:1, from about 15:1 to about 1:1, from about 10:1 to about 1:1, front about 8:1 to about 1:1, from about 6:1 to about 1:1, from about 5:1 to about 1:1, from about 4:1 to about 1:1, from about 3:1 to about 1:1, or from about 2:1 to about 1:1. In certain embodiments, the molar ratio of an iRNA to a crRNA is about 10:1.

In certain embodiments, the molar ratio of an iRNA to a gRNA may range from about 500:1 to about 1:100, 400:1 to about 1:80, 300:1 to about 1:60, 200:1 to about 1:50, 200:1 to about 1:30, from about 200:1 to about 1:20, from about 150:1 to about 1:15, from about 100:1 to about 1:10, from about 80:1 to about 1:5, from about 60:1 to about 1:2, from about 50:1 to about 1:1, from about 40:1 to about 1:1, from about 30:1 to about 1:1, from about 20:1 to about 1:1, from about 15:1 to about 1:1, from about 10:1 to about 1:1, from about 8:1 to about 1:1, from about 6:1 to about 1:1, from about 5:1 to about 1:1, from about 4:1 to about 1:1, from about 3:1 to about 1:1, or from about 2:1 to about 1:1. In certain embodiments, the molar ratio of an iRNA to a gRNA is about 10:1.

In certain embodiments, the molar ratio of an iRNA to a tracrRNA may range from about 500:1 to about 1:100, 400:1 to about 1:80, 300:1 to about 1:60, 200:1 to about 1:50, 200:1 to about 1:30, from about 200:1 to about 1:20, from about 150:1 to about 1:15, from about 100:1 to about 1.:10, from about 80:1 to about 1:5, from about 60:1 to about 1:2, from about 50:1 to about 1:1, from about 40:1 to about 1:1, from about 30:1 to about 1:1, from about 20:1 to about 1:1, from about 15:1 to about 1:1, from about 10:1 to about 1:1, from about 8:1 to about 1:1, from about 6:1 to about 1:1, from about 5:1 to about 1:1, from about 4:1 to about 1:1, from about 3:1 to about 1:1, or from about 2:1 to about 1:1. In certain embodiments, the molar ratio of an iRNA to a tracrRNA is about 10:1.

In certain embodiments, an invader RNA exists as a molecule separate from the single guide RNA (sgRNA) which contains crRNA and tracrRNA. In certain embodiments, an invader RNA exists as a molecule separate from crRNA and tracrRNA. In certain embodiments, an invader RNA can exist as a portion (e.g., the third segment) of a single RNA molecule that also includes the crRNA and tracrRNA. In certain embodiments, the invader RNA can be covalently linked to gRNA, crRNA and/or tracrRNA.

An invader RNA (referred to as “the third segment of the RNA”, “the second RNA”, or “the third RNA” etc. in various embodiments of the present disclosure), can have a length from about 12 nucleotides to about 100 nucleotides. For example, an invader RNA can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, an invader RNA can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. In certain embodiments, an invader RNA has about 10 to about 50 nucleotides, about 12 to about 40 nucleotides, about 14 to about 34 nucleotides, about 18 to about 30 nucleotides, about 20 to about 34 nucleotides, or about 28 to about 34 nucleotides. An invader RNA can have fewer than 12 nucleotides or greater than 100 nucleotides.

An invader RNA (referred to as “the third segment of the RNA”, “the second RNA”, or “the third RNA” etc. in various embodiments of the present disclosure) may comprises 8, 9, 10, 11, 12, 13, 14, 15, 16, 17, 18, 19, 20, 21, 22, 23, 24, 25 or more contiguous nucleotides that have 100% complementarity to a sequence in the non-target strand of a dsDNA, or to a sequence in a DNA or RNA. The nucleotide sequence of the invader RNA that is complementary to a sequence in the non-target strand of a dsDNA, or to a sequence in a DNA or RNA, can have a length of at least about 12 nt. For example, The nucleotide sequence of the invader RNA that is complementary to a sequence in the non-target strand of a dsDNA, or to a sequence in a DNA or RNA, can have a length of at least about 12 nt, at least about 15 nt, at least about 18 nt, at least about 19 nt, at least about 20 nt, at least about 25 nt, at least about 30 nt, at least about 35 nt or at least about 40 nt. For example, The nucleotide sequence of the invader RNA that is complementary to a sequence in the non-target strand of a dsDNA, or to a sequence in a DNA or RNA, can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 from about 20 nt to about 35 nt, from about 20 nt to about 40 nt, from about 20 nt to about 45 nt, from about 20 nt to about 50 nt, from about 20 nt to about 60 nt, or from about 28 nt to about 34 nt.

The percent complementarity between the invader RNA (referred to as “the third segment of the RNA”, “the second RNA”, or “the third RNA” etc. in various embodiments of the present disclosure) and a sequence in the non-target strand of a dsDNA, or a sequence in a DNA or RNA, can be at least 60%, at least 65%, at least 70%, at least 75%, at least 80%, at least 85%, at least 90%, at least 95%, at least 97%, at least 98%, at least 99%, or 100%, over 7, 8, 9, 10, 11, 12, 13, 14, 15, 16, 17, 18, 19, 20, 21, 22, 23, 24, 25 or more contiguous nucleotides.

CrRNA or gRNA (referred to as “the first segment of the RNA”, “the first segment of the first RNA”, or “the first RNA” etc. in various embodiments of the present disclosure) can have a length ranging from about 12 nucleotides to about 100 nucleotides. For example, crRNA or gRNA can have a length ranging from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the first segment (e.g., crRNA) can have a length of from about 19 nt to about 20 nt, from about 19 nt to about 25 nt, from about 19 nt to about 30 nt, from about 19 nt to about 35 nt, from about 19 nt to about 40 nt, from about 19 at to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 at to about 80 nt, from about 20 nt to about 90 nt, or from about 20 nt to about 100 nt. A crRNA or gRNA can have fewer than 12 nucleotides or greater than 100 nucleotides.

CrRNA or gRNA (referred to as “the first segment of the RNA” “the first segment of the first RNA”, or “the first RNA” etc. in various embodiments of the present disclosure) may comprise 5, 6, 7, 8, 9,10, 11, 12, 13, 14, 15, 16, 17, 18, 19, 20, 21, 22, 23, 24, 25 or more contiguous nucleotides that have 100% complementarity to a target sequence in the target nucleic acid (e.g., DNA or RNA). The nucleotide sequence of the crRNA or gRNA that is complementary to a target sequence can have a length of at least about 12 nt. For example, the sequence of crRNA or gRNA that is complementary to a target nucleic acid can have a length of at least about 12 nt, at least about 15 nt, at least about 18 nt, at least about 19 nt, at least about 20 nt at least about 25 nt, at least about 30 nt, at least about 35 nt or at least about 40 nt. For example, the sequence of crRNA or gRNA that is complementary to a target nucleic acid can have a length of from about 12 nucleotides (at) to about 80 at, from about 12 at to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, front about 20 nt to about 45 nt, front about 20 nt to about 50 nt, or from about 20 in to about 60 nt.

The percent complementarity between crRNA or gRNA (referred to as “the first segment of the RNA”, “the first segment of the first RNA”, or “the first RNA” etc. in various embodiments of the present disclosure) and the target nucleic acid (e.g., DNA or RNA) can be at least 60%, at least 65%, at least 70%, at least 75%, at least 80%, at least 85%, at least 90%, at least 95%, at least 97%, at least 98%, at least 99%, or 100%, over 7, 8, 9, 10, 11, 12, 13, 14, 15, 16, 17, 18, 19, 20, 21, 22, 23, 24, 25 or more contiguous nucleotides.

CrRNA (referred to as “the first segment of the RNA”, “the first segment of the first RNA”, or “the first RNA” etc. in various embodiments of the present disclosure) and tracrRNA (referred to as “the second segment of the RNA”, “the second segment of the first RNA”, or “the second RNA” etc. in various embodiments of the present disclosure) may hybridize to form double-stranded RNA duplex (dsRNA duplex), thus resulting in one or more (e.g., 1, 2, 3, 4, 5 or more) stem-loop structures and/or handle structures.

Each segment or each RNA molecule may self-hybridize to form datable-stranded RNA duplex (dsRNA duplex), thus resulting in one or more (e.g., 1, 2, 3, 4, 5 or more) stem-loop structures and/or handle structures.

CrRNA (referred to as “the first segment of the RNA”, “the first segment of the first RNA”, or “the first RNA” etc. in various embodiments of the present disclosure) and tracrRNA (referred to as “the second segment of the RNA”, “the second segment of the first RNA”, or “the second RNA” etc. in various embodiments of the present disclosure) can be covalently linked via the 3′ end of crRNA and the 5′ end of tracrRNA. Alternatively, crRNA and tracrRNA can be covalently linked via the 5′ end of crRNA and the 3′ end of tracrRNA.

TracrRNA (referred to as “the second segment of the RNA”, “the second segment of the first RNA”, or “the second RNA” etc. in various embodiments of the present disclosure) and invader RNA (referred to as “the third segment of the RNA”, “the second RNA”, or “the third RNA” etc. in various embodiments of the present disclosure) can be covalently linked via the 3′ end of tracrRNA and the 5′ end of iRNA. Alternatively, tracrRNA and iRNA can be covalently linked via the 5′ end of tracrRNA and the 3′ end of iRNA.

CrRNA (referred to as “the First segment of the RNA”, “the first segment of the first RNA”, or “the first RNA” etc. in various embodiments of the present disclosure) and invader RNA (referred to as “the third segment of the RNA”, “the second RNA”, or “the third RNA” etc. in various embodiments of the present disclosure) can be covalently linked via the 3′ end of crRNA and the 5′ end of crRNA. Alternatively, crRNA and iRNA can be covalently linked via the 5′ end of crRNA and the 3′ end of iRNA.

CrRNA and iRNA, tracrRNA and iRNA, and/or crRNA and tracrRNA, can be covalently linked by intervening nucleotides (“linker”). The linker can have a length of from about 3 nucleotides to about 100 nucleotides. For example, the linker can have a length of from about 3 nt to about 90 nt, from about 3 nt to about 80 nt, from about 3 nt to about 70 nt, from about 3 nt to about 60 nt, from about 3 in to about 50 nt, from about 3 in to about 40 nt, from about 3 nt to about 30 nt, from about 3 nt to about 20 nt, or from about 3 nt to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, front about 80 nt to about 90 nt, or from about 90 nt to about 100 nt. In some embodiments, the linker is 4 nt.

Also encompassed by the present disclosure are compositions and systems that include or encode the present CRISPR components and/or the Cas9 protein or a variant thereof.

The present disclosure provides for a system that targets a target sequence in a double-stranded DNA, the system comprising: (i) a first DNA polynucleotide encoding a first RNA, the first RNA comprising: (a) a first segment (e.g., crRNA or gRNA) that hybridizes with a target sequence in a target strand of the double-stranded DNA; and (b) a second segment (e.g., tracrRNA) that hybridizes with the first segment to form a double-stranded protein-binding motif; (ii) a second DNA polynucleotide encoding a second RNA (e.g., iRNA), wherein the second RNA hybridizes with a sequence in a non-target strand of the double-stranded DNA; and (iii) a third DNA polynucleotide encoding a Cas enzyme or a variant thereof. In certain embodiments, the first DNA polynucleotide, the second DNA polynucleotide, and the third DNA polynucleotide are within a vector. In certain embodiments, the first DNA polynucleotide, the second DNA polynucleotide, and the third DNA polynucleotide are located on different vectors (e.g., two or three vectors).

The present disclosure provides for a system that targets a target sequence in a double-stranded DNA, the system comprising: (i) a first DNA polynucleotide encoding a first RNA, the first RNA comprising: (a) a first segment (e.g., crRNA or gRNA) that hybridizes with a target sequence in a target strand of the double-stranded DNA; and (b) a second segment (e.g., tracrRNA) that hybridizes with the first segment to form a double-stranded protein-binding motif; and (c) a third segment (e.g., iRNA) that hybridizes with a sequence in a non-target strand of the double-stranded DNA; (ii) a second DNA polynucleotide encoding a Cas enzyme or a variant thereof. In certain embodiments, the first DNA polynucleotide and the second DNA polynucleotide are within a vector. In certain embodiments, the first DNA polynucleotide and the second DNA polynucleotide are located on different vectors.

The present disclosure provides for a system that targets a target sequence in a double-stranded DNA, the system comprising: (i) a first DNA polynucleotide encoding a first RNA (e.g., crRNA or gRNA), wherein the first RNA hybridizes with a target sequence in a target strand of a double-stranded DNA; (ii) a second DNA polynucleotide encoding a second RNA (e.g., tracrRNA), wherein the second RNA hybridizes with the first RNA to form a double-stranded protein-binding motif; (iii) a third DNA polynucleotide encoding a third RNA (e.g., iRNA), wherein the third RNA hybridizes with a sequence in a non-target strand of the double-stranded DNA; and (iv) a fourth DNA polynucleotide encoding a Cas enzyme or a variant thereof, in certain embodiments, the first DNA polynucleotide, the second DNA polynucleotide, the third DNA polynucleotide, and the fourth DNA polynucleotide are within a vector. In certain embodiments, the first DNA polynucleotide, the second DNA polynucleotide, the third DNA polynucleotide, and the fourth DNA polynucleotide are located on different vectors (e.g., 2, 3 or 4 vectors).

The present disclosure provides for a method of targeting a target sequence in a double-stranded DNA, the method comprising the step of contacting the double-stranded DNA with a system comprising: (i) a first RNA, or a DNA polynucleotide encoding a first RNA, wherein the first RNA comprises: (a) a first segment (e.g., crRNA or gRNA) that hybridizes with a target sequence in a target strand of the double-stranded DNA; (b) a second segment (e.g., tracrRNA) that hybridizes with the first segment to form a double-stranded protein-binding motif; (ii) a second RNA (e.g., iRNA), or a DNA polynucleotide encoding a second RNA, wherein the second RNA hybridizes with a sequence in a non-target strand of the double-stranded DNA; and (iii) a Cas enzyme protein or a variant thereof, or a DNA polynucleotide or a RNA polynucleotide encoding a Cas enzyme or a variant thereof.

The present disclosure provides for a method of targeting a target sequence in a double-stranded DNA, the method comprising the step of contacting the double-stranded DNA with a system comprising: (i) a DNA-targeting RNA, or a DNA polynucleotide encoding a DNA-targeting RNA, wherein the DNA-targeting RNA comprises: (a) a first segment (e.g., crRNA or gRNA) that hybridizes with a target sequence in a target strand of the double-stranded DNA; (b) a second segment (e.g., tracrRNA) that hybridizes with the first segment to form a double-stranded protein-binding motif; and (c) a third segment (e.g., iRNA) that hybridizes with a sequence in a non-target strand of the double-stranded DNA; and (ii) a Cas enzyme or a variant thereof, or a DNA polynucleotide or a RNA polynucleotide encoding a Cas enzyme or a variant thereof.

The present disclosure provides for a method of targeting a target sequence in a double-stranded DNA in a cell, the method comprising the step of introducing into the cell a system comprising: (i) a first RNA, or a DNA polynucleotide encoding a first RNA, wherein the first RNA comprises: (a) a first segment (e.g., crRNA or gRNA) that hybridizes with a target sequence in a target strand of the double-stranded DNA; and (b) a second segment (e.g., tracrRNA) that hybridizes with the first segment to form a double-stranded protein-binding motif; and (ii) a second RNA (e.g., iRNA), or a DNA polynucleotide encoding a second RNA, wherein the second RNA hybridizes with a sequence in a non-target strand of the double-stranded DNA.

The present disclosure provides for a method of targeting a target sequence in a double-stranded DNA in a cell, the method comprising the step of introducing into the cell a DNA-targeting RNA, or a DNA polynucleotide encoding a DNA-targeting RNA, wherein the DNA-targeting RNA comprises: (a) a first segment (e.g., crRNA or gRNA) that hybridizes with a target sequence in a target strand of the double-stranded DNA; (b) a second segment (e.g., tracrRNA) that hybridizes with the first segment to form a double-stranded protein-binding motif, and (c) a third segment (e.g., iRNA) that hybridizes with a sequence in a non-target strand of the double-stranded DNA.

In certain embodiments, the cell expresses a Cas enzyme. In certain embodiments, the method further comprises delivering into the cell (i) a DNA polynucleotide encoding a Cas enzyme or a variant thereof, (ii) a RNA polynucleotide encoding a Cas enzyme or a variant thereof, or (iii) a Cas enzyme or a variant thereof.

The present disclosure provides for one or more DNA polynucleotides encoding the present RNAs, CRISPR components, Cas enzymes, etc. The present disclosure also provides for a vector or construct comprising the DNA polynucleotide(s), and a cell comprising the DNA polynucleotide(s) and/or the vector or construct.

The present systems, compositions and methods may modify or alter (e.g., increase or decrease) expression of one or more genes.

Differential gene expression can be achieved by modifying the efficiency of gRNA base-pairing to the target sequence. Larson et al., 2013, CRISPR interference (CRISPRi) for sequence-specific control of gene expression, Nature Protocols 8 (11): 2180-96. Modulating this efficiency may be used to create an allelic series for any given gene, creating a collection of hypomorphs and hypermorphs. These collections can be used to probe any genetic investigation. For hypomorphs, this allows the incremental reduction of gene function as opposed to the binary nature of gene knockouts.

CRISPR interference (CRISPRi) or CRISPR activation (CRISPRa) may he used in the present systems and methods.

CRISPRi is a transcriptional interference technique that allows for sequence-specific repression of gene expression and/or epigenetic modifications in cells. Qi et al., (2013) Repurposing CRISPR as an RNA-guided platform for sequence-specific control of gene expression. Cell 152 (5): 1173.-83. CRISPRi regulates gene expression primarily on the transcriptional level. CRISPRi can sterically repress transcription, e.g., by blocking transcriptional initiation or elongation. The target sequence may be the promoter and/or exonic sequences (such as the non-template strand and/or the template strand), and/or introns. Ji et al., (2014). Specific gene repression by CRISPRi system transferred through bacterial conjugation. ACS Synthetic Biology 3 (12): 929-31. CRISPRi can also repress transcription via an effector domain. Fusing a repressor domain to a catalytically inactive Cas enzyme, e.g., dead Cas9 (dCas9), may further repress transcription. For example, the Krëppel associated box (KRAB) domain can be fused to dCas9 to repress transcription of the target gene. Gilbert et al., 2013, CRISPR-mediated modular RNA-guided regulation of transcription in eukaryotes. Cell 154 (2): 442-51.

CRISPRa utilizes the CRISPR technique to allow for sequence-specific activation of gene expression and/or epigenetic modifications in cells. Qi et al., (2013) Repurposing CRISPR as an RNA-guided platform for sequence-specific control of gene expression, Cell 152 (5): 1173-83. Gilbert et al., (2013) CRISPR-mediated modular RNA-guided regulation of transcription in eukaryotes, Cell 154 (2): 442-51. For example, a catalytically inactive Cas enzyme, e.g., dCas9, may be used to activate genes when fused to transcription activating factors. These factors include, but are not limited to, subunits of RNA Polymerase II and traditional transcription factors, such as VP16, VP64, VPR etc. Gilbert et al., 2014, Genome-Scale CRISPR-Mediated Control of Gene Repression and Activation, Cell 159 (3): 647-61.

The present system may comprise or encode at least one CRISPR RNA (crRNA). The present DNA construct or vector may encode at least one CRISPR RNA (crRNA). in certain embodiments, crRNA contains guide RNA along with a tracrRNA-binding segment which is complementary to at least one portion of a tracrRNA and functions to bind (hybridize to) the tracrRNA and recruit the Cas enzyme to the target sequence.

In the CRISPR system, when the gRNA and the Cas enzyme are expressed, the gRNA directs sequence-specific binding of a CRISPR complex including a Cas enzyme to a target sequence (e.g., coding or non-coding DNA) in the cell. The Cas enzyme may then target (e.g., cleave) the target sequence.

As used herein, a gRNA is complementary to a target nucleic acid sequence in vitro or in a host cell. The gRNA targets the CRISPR/Cas complex to a target nucleic acid sequence, also referred to as a target sequence or a target site.

The gRNA may be between 10-30 nucleotides, 15-25 nucleotides, 15-20 nucleotides, 18-22 nucleotides, or 19-21 nucleotides in length. In some embodiments, a gRNA is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a gRNA is less than about 75, 50, 45, 40, 35, 30, 25, 20, 15, 12, or fewer nucleotides in length. In some embodiments, the gRNA is 20 nucleotides in length.

In general, a gRNA is any polynucleotide sequence having sufficient complementarity with a target sequence to hybridize with the target sequence and direct sequence-specific binding of a CRISPR complex to the target sequence. In some embodiments, the degree of complementarity between a gRNA and its corresponding target sequence, when optimally aligned using a suitable alignment algorithm, is about or more than about 50%, 55%, 60%, 65%, 70%, 75%, 80%, 85%, 90%, 95%. 96%, 97%, 98%, 99%, or 100%. In some embodiments, a gRNA is at least 50%, 55%, 60%, 65%, 70%, 75%, 80%, 85%, 90%, 95%, 96%, 97%, 98%, 99%, or at least 100% complementary to the 3′ end of the target sequence (e.g., the last 5, 6, 7, 8, 9, or 10 nucleotides of the 3′ end of the target sequence).

Each construct or vector of the present system may encode or contain two or more gRNAs. Multiple (two or more) gRNAs can be used to target multiple different genes simultaneously, or target different sites of the same gene.

A tracrRNA-binding segment includes any sequence that has sufficient complementarity with tracrRNA. to promote one or more of: (1) excision of a target sequence targeted by gRNA; and (2) formation of a CRISPR complex at or near a target sequence.

In some embodiments, the degree of complementarity between tracrRNA and tracrRNA-binding segment is about or more than about 25%, 30%, 40%, 50%, 60%, 65%, 70%, 75%, 80%, 85%, 90%, 95%, 97.5%, 99%, or 100%, along the length of the shorter of the two when optimally aligned, e.g., over a stretch of at least 8 contiguous, at least 9 contiguous, at least 10 contiguous, at least 11 contiguous, at least 12 contiguous, at least 13 contiguous, at least 14 contiguous or at least 15 contiguous nucleotides.

Exemplary tracrRNA-binding segment sequences can be found, for example, in Jinek, et al. Science (2012) 337(6096):816-821; Ran, et al. Nature Protocols (2013) 8:2281-2308; WO2014/093694; WO2013/176772 and WO2016070037.

TracrRNA-binding segment sequences may be wildtype or mutated.

The present system (a construct or a vector) may contain or encode a tracrRNA.

A trans-activating crRNA (tracrRNA) refers to an RNA that recruits a Cas enzyme to a target sequence bound (hybridized) to a complementary crRNA.

In some embodiments, the tracrRNA is about or more than about 5, 6, 7, 8, 9, 10, 11, 12, 13, 14, 15, 16. 17, 18, 19, 20, 25, 26, 30, 32, 40, 45, 48, 50, 54, 63, 67, 85, or more nucleotides in length.

In some embodiments, the tracrRNA has sufficient complementarity to a tracrRNA-binding segment of crRNA to hybridize and participate in the formation of a CRISPR complex.

TracrRNA sequences may be wildtype or mutated.

In certain embodiments, crRNA (containing gRNA) and tracrRNA are expressed as separate transcripts. The present system (a construct or a vector) may contain or express crRNA and tracrRNA as separate transcripts.

In certain embodiments, crRNA (containing gRNA and tracrRNA-binding segment) and tracrRNA are contained within a single transcript (e.g., sgRNA). The present system (a construct or a vector) may contain or express sgRNA.

A single guide RNA (sgRNA) is a chimeric RNA containing a tracrRNA and at least one crRNA (containing gRNA). An sgRNA has the dual function of both binding (hybridizing) to a target sequence and recruiting the Cas enzyme to the target sequence.

CrRNA and tracrRNA can be covalently linked via the 3′ end of the crRNA and the 5′ end of the tracrRNA. Alternatively, crRNA and tracrRNA Can be covalently linked via the 5′ end of the crRNA and the 3′ end of the tracrRNA.

In such embodiments, sgRNA may have a secondary structure, such as a hairpin. In certain embodiments, the transcript or transcribed polynucleotide sequence has at least two or more hairpins. For Example, the transcript may have two, three, four, five, or more than five hairpins. SgRNA may comprise a tinker loop structure and/or a stem-loop structure. sgRNA used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In some embodiments, sgRNA can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

To facilitate sgRNA design, many computational tools have been developed (See Prykhozhij et al. (PLoS ONE, 10(3): (2015)); Zhu et al. (PLoS ONE, 9(9) (2014)); Xiao et al. (Bioinformatics. Jan. 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there is a publically available software tool that can be used to facilitate the design of sgRNA(s) (http://www.genscript.com/gRNA-design-tool.html).

SgRNA sequences may be wildtype or mutated.

Chimeric RNA may be used to refer to a fusion of at least the tracrRNA-binding segment and tracrRNA. Chimeric RNA sequences may be wildtype or mutated.

The present system (e.g., constructs, vectors, cells, etc.) may or may not mode a Cas enzyme.

The Cas enzyme targets (e.g., cleaves) of one or two strands at or near a target sequence, such as within the target sequence and/or within the complementary strand of the target sequence. For example, the Cas enzyme may target (e.g., cleave) of one or both strands within about 1, 2, 3, 4, 5, 6, 7, 8, 9, 10, 15, 20, 25, 50, 100, 200, 500, or more nucleotides from the first or last nucleotide of a target sequence. In certain embodiments, formation of a CRISPR complex results in cleavage (e.g., a cutting or nicking) of one or both strands in or near (e.g. within 1, 2, 3, 4, 5, 6, 7, 8, 9, 10, 20, 50, or more base pairs from) the target sequence. In some embodiments, the Cas enzyme lacks DNA strand cleavage activity.

The Cas enzyme may be a type II, type I, type III, type IV or type V CRISPR system enzyme. In some embodiments, the Cas enzyme is a Cas9 enzyme (also known as Csn1 and Csx12).

Cas9 may be wild-type or mutant. Cas9 may be any variant disclosed in U.S. Patent Publication No. 2014/0068797. Cas9 may be Type II-A, Type II-B, or Type II-C. Cas9 may be from various species, including, but not limited to, S. pyogenes, N. meningitides, C. jejuni, R. palustris, R. Rubrum, A. naeslundii and C. diphtheria.

In some embodiments, the Cas9 is a modified form or a variant of the wild-type Cas9. In some instances, the modified form of the Cas9 protein comprises an amino acid change (e.g., deletion, insertion, or substitution) that reduces the naturally-occurring nuclease activity of the Cas9 protein. In certain embodiments, the modified form of the Cas9 protein has less than SO%, less than 40%, less than 30%, less than 20%, less than 10%, less than 5%, or less than 1% of the nuclease activity of the corresponding wild-type Cas9 protein. In some cases, the modified form of the Cas9 protein has no substantial nuclease activity.

In some embodiments, the Cas9 protein can be codon-optimized.

Non-limiting examples of the Cas9 enzyme include Cas9 derived from Streptococcus pyogenes (S. pyogenes), S. pneumoniae, Staphylococcus aureus, Neisseria meningitidis, Streptococcus thermophilus (S. thermophilus), or Treponema denticola. The Cas enzyme may also be derived from Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter.

Non-limiting examples of the Cas enzymes also include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, orthologs thereof, or modified versions thereof.

One or more of the CRISPR components nod a Cas enzyme may be encoded by the same construct (e.g., a vector). Alternatively, a Cas enzyme may be encoded by a construct (e.g., a vector) separate from the vector encoding one or more of the other CRISPR components. In some embodiments, the present system comprises two or more Cas enzyme coding sequences operably linked to different promoters. In some embodiments, the host cell expresses one or more Cas enzymes.

The Cas enzyme can be introduced into a cell in the form of a DNA, mRNA or protein. The Cas enzyme may be engineered, chimeric, or isolated from an organism.

Wildtype or mutant Cas enzyme may be used. In some embodiments, the nucleotide sequence encoding the Cas9 enzyme is modified to alter the activity of the protein. The mutant Cas enzyme may lack the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, D10A, H840A, N854A, N863A, and combinations thereof. In some embodiments, a Cas9 nickase may be used in combination with guide RNA(s), e.g., two guide RNAs, which target respectively sense and antisense strands of the DNA target.

Two or more catalytic domains of Cas9 (RuvC and/or HNH domains) may be mutated to produce a mutated Cas9 substantially lacking all DNA cleavage activity (a catalytically inactive Cas9). In some embodiments, a D10A mutation is combined with one or more of H840A, N854A, or N863A. mutations to produce a Cas9 enzyme substantially lacking DNA cleavage activity (dead Cas 9 or dCas9). In some embodiments, a Cas enzyme is considered to substantially lack DNA cleavage activity when the DNA cleavage activity of the mutated enzyme is about or less than about 25%, 10%, 5%, 1%, 0.1%, 0.01%, or lower, compared to its non-mutated (wildtype) form. Other mutations may be useful; where the Cas9 or other Cas enzyme is from a species other than S. pyogenes, mutations in corresponding amino acids may be made to achieve similar effects.

Another Cas enzyme, Cpf1 (Cas protein 1 of PreFran subtype) may also be used in the present systems and methods. Zetsche et al. Cell, 163 (3): 759-771. In one embodiment, CRISPR-Cpf1 system can be used to cleave a desired region at or near a target sequence. A Cpf1 nuclease may be derived from Pwvetella spp., Francisella spp., etc.

Alternatively or in addition, the Cas enzyme may be fused to another protein or portion thereof. In some embodiments, dCas9 is fused to a repressor domain, such as a KRAB domain. In some embodiments, such dCas9 fusion proteins are used with the constructs described herein for multiplexed gene repression (e.g., CRISPR interference (CRISPRi)). In some embodiments, dCas9 is fused to an activator domain, such as VP64 or VPR. In some embodiments, such dCas9 fusion proteins are used with the constructs described herein for multiplexed gene activation (e.g. CRISPR activation (CRISPRa)).

In some embodiments, dCas9 is fused to an epigenetic modulating domain, such as a histone demethylase domain or a histone acetyltransferase domain. In some embodiments, dCas9 is fused to a LSD1 or p300, or a portion thereof. In some embodiments, the dCas9 fusion is used for CRISPR-based epigenetic modulation.

In some embodiments, dCas9 or Cas9 is fused to a Fok1 nuclease domain. In some embodiments, Cas9 or dCas9 fused to a Fok1 nuclease domain is used for multiplexed gene editing.

In some embodiments, Cas9 or dCas9 is fused to a fluorescent protein (e.g., GFP, RFP, mCherry, etc.), for, e.g., multiplexed labeling and/or visualization of genomic loci.

A sequence encoding a Cas enzyme may be codon-optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. In some embodiments, one or more codons (e.g. 1, 2, 3, 4, 5, 10, 15, 20, 25, 50, or more, or all codons) in a sequence encoding a Cas enzyme correspond to the most frequently used codon for a particular amino acid in the host cell.

The Cas enzyme may be a part of a fusion protein comprising one or more heterologous protein domains (e.g. about or more than about 1, 2, 3, 4, 5, 6, 7, 8, 9, 10, or more domains in addition to the Cas enzyme). A Cas enzyme fusion protein may comprise any additional protein sequence, and optionally a linker sequence between any two domains. Examples of protein domains that may be fused to a Cas enzyme include, without limitation, epitope tags, reporter proteins (or reporters), and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). The sequence encoding a Cas enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP 16 protein fusions. U.S. Patent Publication No. 20110059502. WO2015065964. In some embodiments, a tagged Cas enzyme is used to identify the location of a target sequence.

The Cas enzyme may contain one or more nuclear localization sequences (NLS).

The present construct (e.g., a vector) may contain one, two or more enzyme-coding sequences. The two or more enzyme-coding sequences may comprise two or more copies of a single enzyme-coding sequence, two or more different enzyme-coding sequences, or combinations of these. In such an arrangement, the two or more enzyme-coding sequences may be operably linked to a promoter or to different promoters in a single vector or in multiple vectors. For example, a single vector, or multiple vectors, may comprise about or more than about 1, 2, 3, 4, 5, 6, 7, 8, 9, 10, 15, 20, or more enzyme-coding sequences. In some embodiments, about or more than about 1, 2, 3, 4, 5, 6, 7, 8, 9, 10, or more such enzyme-coding sequence-containing vectors may be provided, and optionally delivered to a cell.

A target sequence refers to any nucleic acid sequence in a host cell that may be targeted by the present systems. A CRISPR gRNA may be selected to target any target sequence in, e.g., a double-stranded DNA (dsDNA), a single-stranded DNA (ssDNA), a double-stranded RNA (dsRNA), and/or a single-stranded RNA (ssRNA). In certain embodiments, the target sequence is a sequence within a genome of an organism.

In certain embodiments, the target sequence is not flanked downstream (on the 3′ side) or upstream (on the 5′ side) by a protospacer adjacent motif (PAM). In certain embodiments, the target sequence is flanked downstream (on the 3′ side) or upstream (on the 5′ side) by a PAM.

The sequence and length requirements for the PAM differ depending on the Cas enzyme used. PAMs may be 2-8 base pair sequences adjacent the target sequence. In certain embodiments, the PAM is a 2-6 base pair DNA sequence immediately following the DNA sequence targeted by the Cas nuclease in the CRISPR system. It was reported that Cas9 would not successfully bind to or cleave the target DNA sequence if it was not followed by the PAM sequence. Mojica et al., (2009) Short motif sequences determine the targets of the prokaryotic CRISPR defense system, Microbiology, 155 (Pt 3): 733-740, Shah et al., (2013) Protospacer recognition motifs: mixed identities and functional diversity, RNA Biology, 10 (5); 891-899. Jinek et al., (2012) A programmable dual-RNA-guided DNA endonuclease in adaptive bacterial immunity, Science, 337 (6096): 816-821. Sternberg et al., (2014) DNA interrogation by the CRISPR RNA-guided endonuclease Cas9, Nature, 507 (7490): 62-67. PAM was considered an essential targeting component (not found in bacterial genome) which distinguishes bacterial self from non-self DNA, thereby preventing the CRISPR locus from being targeted and destroyed by nuclease. Mali et al., (2013) Cas9 as a versatile tool for engineering biology, Nature Methods, 10 (10): 957-963.

For example, for Cas9 endonucleases derived from Streptococcus pyogenes (S. pyogenes), the PAM sequence is NGG. For Cas9 endonucleases derived from Staphylococcus aureus, the PAM sequence is NNGRRT. For Cas9 endonucleases derived from Neisseria meningitidis, the PAM sequence is NNNNGATT. For Cas9 endonucleases derived from Streptococcus thermophilus, the PAM sequence is NNAGAA. For Cas9 endonuclease derived from Treponema denticola, the PAM sequence is NAAAAC. For a Cpf1 nuclease, the PAM sequence is TTN.

A target sequence may be located in the nucleus or cytoplasm of a cell. The target sequence may be within an organelle of a eukaryotic cell, for example, mitochondrion or chloroplast. The target sequence can be a sequence encoding a gene product (e.g., a protein) or a non-coding sequence (e.g., a regulatory polynucleotide or a junk DNA). A target sequence may be endogenous (endogenous to the cell) or exogenous (exogenous to the cell) sequences. A target sequence may be genomic nucleic acid and/or extra-genomic nucleic acid.

A target sequence may be a nucleic acid encoding transcription factors, signaling proteins, transporters, epigenetic genes, etc. A target sequence may be, or contains part(s) of, constitutive exons downstream of a start codon of a gene. A target sequence may be, or contains part(s) of, either a first or a second exon of a gene. In one embodiment, the target sequence is a transcribed or non-transcribed strand of a gene.

A target sequence may be a part of gene regulatory sequences such as promoters and transcriptional enhancer sequences, ribosomal binding sites and other sites relating to the efficiency of transcription, translation, or RNA processing, as well as coding sequences that control the activity, post-translational modification, or turnover of the encoded proteins. U.S. Patent Publication No. 20160186168.

A target sequence may be parts of one or more disease-associated genes and polynucleotides as well as signaling pathway-associated genes and/or polynucleotides. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

A target sequence may be part(s) of one or more genes and/or polynucleotides relating to a particular pathway (for example, an enzymatic pathway, an immune pathway or a cell division pathway), or a particular disease or group of diseases or disorders (e.g., cancer). U.S. Patent Publication No. 20150064138.

For example, a target sequence may be part(s) of one or more genes and/or polynucleotides associated with epigenetic changes in cancer, diabetes, obesity, neurological disorders (e.g., schizophrenia), or function in processes such as aging.

In one embodiment, the target sequence may be part(s) of one or more genes and/or polynucleotides described in Kazuhiro et al., Epigenetic clustering of gastric carcinomas based on DNA methylation profiles at the precancerous stage: its correlation with tumor aggressiveness and patient outcome, Carcinogenesis, 2015, Vol. 36, No. 5, 509-520.

The present molecules, systems and methods may target one or more target sequences in one or more genes and/or polynucleotides, such as about, or more than about, 2, 3, 4, 5, 6, 7, 8, 9, 10, 20, or more genes and; or polynucleotides.

The target sequences may be different loci within the same gene(s). The target sequences may be different genes and/or polynucleotides. The present molecules, systems and methods may target 2 to 20 or more different loci within the same gene or across multiple genes. For example, the present molecules, systems and methods may target 1, 2, 3, 4, 5, 6, 7, 8, 9, 10, 11, 12, 13, 14, 15, 16, 17, 18, 19, 20, or more different target sequences.

The present system may contain one or more regulatory elements that are operably linked to one or more elements of the present CRISPR system so as to drive expression of the one or more elements of the present CRISPR system.

Regulatory elements may include promoters, enhancers, activator sequences, and other expression control elements (e.g. transcription termination signals, such as polyadenylation signals and poly-U sequences). The vectors of the invention may optionally include 5′ leader or signal sequences. Such regulatory elements are described, for example, in Goeddel, Gene Expression Technology: Methods in Enzymology, Academic Press (1990). A tissue-specific promoter may direct expression primarily in a desired tissue of interest. Regulatory elements may direct expression in a tissue-specific, cell-type specific, and/or a temporal-dependent manner, such as in a cell-cycle dependent or developmental stage-dependent manner.

In some embodiments, a vector comprises one or more pol III promoter (e.g. 1, 2, 3, 4, 5, or more pol III promoters), one or more pol II promoters (e.g. 1, 2, 3, 4, 5, or more pol II promoters), such as a mammalian RNA polymerase II promoter, one or more pol I promoters (e.g. 1, 2, 3, 4, 5, or more pol I promoters), or combinations thereof.

Examples of pol III promoters include, but are not limited to, H1 promoter, U6 promoter, mouse U6 promoter, swine U6 promoter. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV4G promoter, the dihydro folate reductase promoter, the β-actin promoter, the phosphoglycerol kinase (PGK) promoter, and the EF1 a promoter. Boshart et al, Cell, 41: 521-530 (1985). In some embodiments, the promoter is a human ubiquitin C promoter (UBCp). In some embodiments, the promoter is a viral promoter. In some embodiments, the promoter is a human cytomegalovirus promoter (CMVp).

Non-limiting examples of enhancers include WPRE; CMV enhancers; the R-U5′ segment in LTR of HTLV-1 (Mol. Cell. Biol, Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

The present vector may contain one or more promoters upstream of the sequence encoding iRNA, the sequence encoding isgRNA, the sequence encoding gRNA, the sequence encoding crRNA, the sequence encoding tracrRNA, the sequence encoding sgRNA, the sequence encoding the chimeric RNA (containing tracrRNA-binding segment and tracrRNA), and/or the sequence encoding a Cas enzyme.

As used herein, the terms “under the control”, “under transcriptional control”, “operably positioned”, and “operably linked” mean that a promoter is in a correct functional location and/or orientation in relation to a nucleic acid sequence, a DNA fragment, or a gene, to control transcriptional initiation and/or expression of that sequence, DNA fragment or gene.

The promoter may be constitutive, regulatable or inducible; cell type-specific, tissue-specific, or species-specific.

A constitutive promoter is an unregulated promoter that allows for continual transcription of the gene under the promoter's control. Many promoter/regulatory sequences useful for driving constitutive expression of a gene are available in the art and include, but are not limited to, for example, U6 (human U6 small nuclear promoter), H1 (human polymerase III RNA promoter), CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), and the like.

Sequences encoding the present CRISPR component (e.g., a sequence encoding an iRNA, a sequence encoding an isgRNA, a sequence encoding a gRNA, a sequence encoding an sgRNA, a sequence encoding a chimeric RNA, a sequence encoding a crRNA, a sequence encoding a tracrRNA, a sequence encoding a Cas enzyme) may be under the control of an inducible promoter or a constitutive promoter.

The transcriptional activity of inducible promoters may be induced by chemical or physical factors. Chemically-regulated inducible promoters may include promoters whose transcriptional activity is regulated by the presence or absence of oxygen, a metabolite, alcohol, tetracycline, steroids, metal and other compounds. Physically-regulated inducible promoters, including promoters whose transcriptional activity is regulated by the presence or absence of heat, low or high temperatures, acid, base, or light. In one embodiment, the inducible promoter is pH-sensitive (pH inducible). The inducer for the inducible promoter may be located in the biological tissue or environmental medium to which the composition is administered or targeted, or is to be administered or targeted. Examples of tissue specific or inducible promoter/regulatory sequences include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters can be found at http://www.invivogen.com/prom-a-list. In addition, promoters which can be induced in response to inducing agents such as metals, glucocorticoids, tetracycline, hormones, and the like, are also contemplated for use with the present systems and methods. The pH level of a particular biological tissue can affect the inducibility of the pH inducible promoter. See, for example, Boron, et al., Medical Physiology: A Cellular and Molecular Approach. Elsevier/Saunders. (2004), ISBN 1-4160-2328-3. Examples of inducers that can induce the activity of the inducible promoters also include, but are not limited to, doxycycline, radiation, temperature change, alcohol, antibiotic, steroid, metal, salicylic acid, ethylene, benzothiadiazole, or other compound. In an embodiment, the at least one inducer includes at least one of arabinose, lactose, maltose, sucrose, glucose, xylose, galactose, rhamnose, fructose, melibiose, starch, inunlin, lipopolysaccharide, arsenic, cadmium, chromium, temperature, light, antibiotic, oxygen level, xylan, nisin, L-arabinose, allolactose, D-glucose, D-xylose, D-galactose, ampicillin, tetracycline, penicillin, pristinamycin, retinoic acid, or interferon. Other examples of inducers include, but are not limited to, clathrate or caged compound, protocell, coacervate, microsphere, Janus particle, proteinoid, laminate, helical rod, liposome, macroscopic tube, niosome, sphingosome, vesicular tube, vesicle, unilarnellar vesicle, multilamellar vesicle, multivesicular vesicle, lipid layer lipid bilayer, micelle, organelle, nucleic acid, peptide, polypeptide, protein, glycopeptide, glycolipid, lipoprotein, lipopolysaccharide, sphingolipid, glycosphingolipid, glycoprotein, peptidoglycan, lipid, carbohydrate, metalloprotein, proteoglycan, chromosome, nucleus, acid, buffer, protic solvent, aprotic solvent, nitric oxide, vitamin, mineral, nitrous oxide, nitric oxide synthase, amino acid, micelle, polymer, copolymer, monomer, prepolymer, cell receptor, adhesion molecule, cytokine, chemokine, immunoglobulin, antibody, antigen, extracellular matrix, cell ligand, zwitterionic material, cationic material, oligonucleotide, nanotube, piloxymer, transfersome, gas, element, contaminant, radioactive particle, radiation, hormone, virus, quantum dot, temperature change, thermal energy, or contrast agent. Theys, et al., Abstract, Curt. Gene Ther. vol. 3, no. 3 pp. 207-221 (2003),

Each of the present constructs (or vectors) may contain one or more sequences encoding one of more CRISPR components. The sequences may encode two or more copies of a CRISPR iRNA or isgRNA, two or more different CRISPR iRNAs or isgRNAs, or combinations thereof.

The sequences encoding the two or more CRISPR components may be operably linked to the same promoter or linked to different promoters. For example, the sequences encoding the two or more CRISPR components may be operably linked to two or more promoters. In one embodiment, sequences encoding two CRISPR components are operably linked to two promoters; thus two transcripts would be transcribed. In another embodiment, sequences encoding two CRISPR components are operably linked to a promoter; thus one transcript would be transcribed.

The two or more promoters may take any suitable position and/or orientation. For example, the two or more promoters may be unidirectional or bidirectional.

The forward primer and/or reverse primer may or may not contain at least one restriction site for cloning at a later stage. The restriction site can be specific to any suitable restriction enzymes, such as Type I, II or III restriction enzymes. Other types of restriction enzymes can also be used, including, but not limited to, Type IIS restriction endonucleases (e.g., Golden Gate Assembly, New England Biolabs).

The two or more promoters of the present system may take suitable position and/or orientation. For example, the two or more promoters may be unidirectional or bidirectional.

The present system (e.g., the present constructs, vectors, etc.) driving expression of one or more elements of a CRISPR system may be introduced into a population of cells to target one or more target sequences.

In certain embodiments, a sequence encoding a Cas enzyme and a sequence encoding one or more CRISPR components are operably linked to separate promoters on separate vectors. Alternatively, a sequence encoding a Cas enzyme and a sequence encoding one or more CRISPR components are operably linked to separate promoters on a single vector.

The sequences encoding the CRISPR components that are combined in a single vector may be arranged in any suitable orientation, such as one element located 5′ with respect to (upstream of) or 3′ with respect to (downstream of) a second element. The coding sequence of one element may be located on the same or opposite strand of the coding sequence of a second element, and oriented in the same or opposite direction.

In some embodiments, the present system (e.g., a vector, a construct, etc.) comprises: (a) at least one first promoter operably linked to one or more sequences encoding one or more CRISPR components; (b) at least one second promoter operably linked to one or more sequences encoding one or more CRISPR components: and (c) at least one third promoter operably linked to a sequence encoding a Cas enzyme.

In some embodiments, a single promoter drives expression of a transcript encoding a Cas enzyme, a sequence encoding an sgRNA (crRNA-tracrRNA), and a sequence encoding an iRNA. In some embodiments, a sequence encoding a Cas enzyme, a sequence encoding an sgRNA (crRNA-tracrRNA) and a sequence encoding an iRNA, are operably linked to and expressed from two or more promoters.

In some embodiments, a single promoter drives expression of a transcript encoding a Cas enzyme, and a sequence encoding an isgRNA (sgRNA covalently linked to an iRNA). In some embodiments, a sequence encoding a Cas enzyme, a sequence encoding an isgRNA (sgRNA covalently linked to an iRNA), are operably linked to and expressed from two or more promoters.

In some embodiments, a vector comprises one or more insertion sites, such as a restriction recognition site (also referred to as a restriction site, or a cloning site). One or more insertion sites (e.g. about or more than about 1, 2, 3, 4, 5, 6, 7, 8, 9, 10, or more insertion sites) may be located upstream and/or downstream of one or more sequences encoding one or more CRISPR components.

In some embodiments, a vector comprises one or more insertion sites upstream of a sequence encoding a tracrRNA-binding segment, and/or a sequence encoding a tracrRNA, and/or a sequence encoding a chimeric RNA or an sgRNA. In some embodiments, a vector comprises one or more insertion sites downstream of a sequence encoding a tracrRNA-binding segment, and/or a sequence encoding a tracrRNA, and/or a sequence encoding a chimeric RNA or an sgRNA.

In some embodiments, a vector comprises an insertion site downstream of a promoter. In some embodiments, a vector comprises one or more insertion sites (e.g. about or more than about 1, 2, 3, 4, 5, 6, 7, 8, 9, 10, or more insertion sites) downstream of a promoter.

One or more sequences encoding one or more CRISPR components and the sequence encoding a Cas enzyme may be located on the same or different vectors.

In some embodiments, sequences encoding one or more of the present CRISPR components are part of a vector system transiently transfected into the host cell. Alternatively or additionally, sequences encoding one or more of the present CRISPR components are stably integrated into a genome of a host cell.

A single vector, or two or more vectors, may be used to target CRISPR activity to, one, two, or more different target sequences in vitro or within a cell. For example, a single vector, or two or more vectors, may encode one or more of the present CRISPR components targeting about, or more than about, 1, 2, 3, 4, 5, 6, 7, 8, 9, 10, 15, 20, or more target sequences. In some embodiments, about, or more than about, 1, 2, 3, 4, 5, 6, 7, 8, 9, 10, or more vectors encoding one or more of the present CRISPR components targeting about, or more than about, 1, 2, 3, 4, 5, 6, 7, 8, 9, 10, or more target sequences may be provided, and optionally delivered to a population of cells. U.S. Patent Publication No. 20150133315.

A single vector, or two or more vectors, may be used to encode one, two, or more different iRNAs or isgRNAs. For example, a single vector, or two or more vectors, may encode about, or more than about, 1, 2, 3, 4, 5, 6, 7, 8, 9, 10, 15, 20, or more iRNAs or isgRNAs. In some embodiments, about, or more than about, 1, 2, 3, 4, 5, 6, 7, 8, 9, 10, or more vectors encoding about, or more than about, 1, 2, 3, 4, 5, 6, 7, 8, 9, 10, or more iRNAs or isgRNAs may be provided, and optionally delivered to a population of cells.

As used herein, a “vector” may be any of a number of nucleic acids into which a desired sequence or sequences may be inserted for transport between different genetic environments or for expression in a host cell. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art.

Vectors include, but are not limited to, viral vectors, plasmids, cosmids, fosmids, phages, phage lambda, phagemids, and artificial chromosomes.

Viral vectors may be derived from DNA viruses or RNA viruses, which have either episomal or integrated genomes after delivery to the cell. Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al in Current Topics in Microbiology and Immunology, Doerfler and Bohm (eds) (1995); and Yu et al., Gene Therapy 1:13-26 (1994).

Viral vectors may be derived from retroviruses (including lentiviruses), replication defective retroviruses (including replication defective lentiviruses), adenoviruses, replication defective adenoviruses, adeno-associated viruses (AAV), herpes simplex viruses, and poxviruses. In some embodiments, the vector is a lentiviral vector. Options for gene delivery of viral constructs are known (see, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71).

Any subtype, serotype and pseudotype of lentiviruses, and both naturally occurring and recombinant forms, may be used as a vector for the present systems and methods. Lentiviral vectors may include, without limitation, primate lentiviruses, goat lentiviruses, sheep lentiviruses, horse lentiviruses, cat lentiviruses, and cattle lentiviruses.

The term AAV covers all subtypes, serotypes and pseudotypes, and both naturally occurring and recombinant forms. AAV viral vectors may be selected from among any AAV serotype, including, without limitation AAV1, AAV2, AAV3, AAV4, AAV5, AAV6, AAV7, AAV8, AAV9, AAV10 or other known and unknown AAV serotypes. Pseudotyped AAV refers to an AAV that contains capsid proteins from one serotype and a viral some of a second serotype.

A variety of vectors may be used to deliver CRISPR components to the targeted cells and/or a subject. In some embodiments, one or more sequences encoding one or more of the present CRISPR components are part of the same vector, or two or more vectors.

The constructs encoding the present CRISPR components can be delivered to the subject using one or more vectors (e.g., 1, 2, 3, 4, 5, 6, 7, 8, 9, or more vectors). One or more sequence encoding one or more CRISPR components can be packaged into a vector. A Cas enzyme can be packaged into the same, or alternatively separate, vectors.

Vectors may further contain one or more marker sequences suitable for use in the identification of cells which have or have not been infected, transformed, transduced or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein, red fluorescent protein). Molecular Cloning: A Laboratory Manual, Fourth Edition, Cold Spring Harbor Laboratory Press, 2012. Current Protocols in Molecular Biology, John Wiley & Sons, Inc.

Vectors can be designed for expression of CRISPR components in prokaryotic or eukaryotic cells. For example, CRISPR transcripts can be expressed in bacterial cells such as Escherichia coli, insect cells (using baculovirus expression vectors), yeast cells, or mammalian cells. Alternatively, one or more sequences encoding one or more of the CRISPR components can be transcribed and translated in vitro.

Vectors may be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system).

In some embodiments, a prokaryote is used to amplify copies of a vector and express one or more nucleic acids, such as to provide a source of one or more proteins for delivery to a host cell or host organism. In some embodiments, a vector is a yeast expression vector. In some embodiments, a vector drives protein expression in insect cells using baculovirus expression vectors.

In some embodiments, a vector is capable of driving expression of one or more sequences in mammalian cells using a mammalian expression vector. Non-limiting examples of promoters include those derived from polyoma, adenovirus 2, cytomegalovirus, simian virus 40, and others disclosed herein and known in the art. Sambrook, et al., Molecular Cloning: A Laboratory Manual, 4th ed., Cold Spring Harbor Laboratory, Cold Spring Harbor Laboratory Press, Cold Spring Harbor, N.Y., 2012.

Reporter genes that may be used with the present systems and methods include, but are not limited to, sequences encoding glutathione-S-transferase (GST), horseradish peroxidase (HRP), chloramphenicol acetyltransferase (CAT) beta-galactosidase, luciferase, green fluorescent protein (GFP), cyan fluorescent protein (CFP), yellow fluorescent protein (YFP), and auto fluorescent proteins including blue fluorescent protein (BFP), may be introduced into a cell to encode a gene product which serves as a marker.

In some embodiments, sequences encoding one or more of the present CRISPR components may contain modifications including, but not limited to, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (e.g., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, historic acetyltransferases, histone deacetylases, and the like); and combinations thereof.

The present disclosure also provides for libraries comprising two or more of the present constructs (e.g., vectors), or two or more of gRNAs. A library of constructs (e.g., vectors) refers to a collection of two or more constructs (e.g., vectors).

The present disclosure provides a library of gRNAs with corresponding iRNAs or isgRNAs. The present disclosure provides a library of nucleic acids (e.g., constructs, vectors, etc.) encoding gRNAs and corresponding iRNAs or isgRNAs. For example, the present library may be a vector library encoding gRNAs and corresponding iRNAs or isgRNAs.

The present system may be a genome wide library. The library may target a subset of the genome of an organism, or a set of genes relating to a particular pathway or phenotype. The set of genes targeted by the present system may be the entire genome of an organism, or may be a subset of the genome of an organism. The set of genes may relate to a particular pathway (for example, an enzymatic pathway, an immune pathway or a cell division pathway) or a particular disease or group of diseases or disorders (e.g., cancer) may be selected.

The present library may target about 100 or more sequences, about 1000 or more sequences or about 20,000 or more sequences, or the entire genome of an organism. The target sequences may be different loci within the same gene(s). The target sequences may be different genes. The present library may target 2 to 60 different loci within the same gene target or across multiple gene targets. For example, the present library may target 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40.41, 42, 43, 44, 45, 46, 47, 48.49, 50, 51, 52, 53, 54, 55, 56, 57, 58, 59 or 60 different DNA sequences. In some embodiments, the present library may target more than 60 different loci within the same gene target or across multiple gene targets, such as 65, 70, 75, 80, 85, 90, 95, 100 or more different DNA sequences.

The library may alter (decrease or increase) the expression level or the function of at least one gene, e.g., all genes of the set of genes. The library may result in a knockout of at least one gene, e.g., all genes of the set of genes.

The present system (e.g., libraries, constructs, vectors) and method may reduce (or increase) the expression level of at least one gene by at least 10%, 15%, 20%, 25%, 30%, 35%, 40%, 45%, 50%, 55%, 60%, or at least 65% as compared to expression level of the gene in the absence of the present system. The present system (e.g., the present library) may reduce activity of at least one protein encoded by a gene by at least 10%, 15%, 20%, 25%, 30%, 35%, 40%, 45%, 50%, 55%, 60%, or at least 65% as compared to activity of the protein encoded by the gene in the absence of the present system.

DNA may be isolated from cells by any method well known in the art. For example, DNA extraction may include two or more of the following steps: cell lysis, addition of a detergent or surfactant, addition of protease, addition or RNase, alcohol precipitation (e.g., ethanol precipitation, or isopropanol precipitation), salt precipitation, organic extraction (e.g., phenol-chloroform extraction), solid phase extraction, silica gel membrane extraction, Csa gradient purification. Various commercial kits (e.g., kits of Qiagen, Valencia, Calif.) can be used to extract DNA.

The DNA fragments may or may not be separated by gel electrophoresis prior to insertion into vectors.

DNA fragments may be inserted into vectors using, e.g., DNA ligase. Each vector may contain a different insert of DNA. in some embodiments, fragmented DNA is end-repaired before being ligated to a vector. Fragmented DNAs may be ligated to adapters before being inserted into vectors.

This present system (libraries, constructs, vectors, etc.) may be used for screening genetic interactions, gene functions, etc. in cellular processes as well as diseases.

A library may be introduced into a population of cells in vitro or in vivo to screen for beneficial mutations (or combinations of mutations) in a set of genes, and a desired phenotype identified. The set of genes may be the entire genome of an organism, a subset of the genome of an organism, or genes involved in target pathways (e.g., a metabolic pathway, a signaling pathway, etc.).

The present disclosure also provides for a method of mapping genetic interactions by delivering the present system (e.g., the present libraries, constructs, vectors) into a population of cells.

The present disclosure also provides for methods of delivering one or more nucleic acids (e.g., the present systems, constructs, vectors, libraries etc., and/or one or more of the present CRISPR components), one or more transcripts thereof, and/or one or proteins transcribed therefrom, to a population of cells.

The present system may also encode a Cas enzyme, such as a Cas9. The present method may also include delivering DNA or mRNA encoding a Cas enzyme to the cells. Alternatively or additionally, the cells may express a Cas enzyme (e.g., Cas9 expressing cells). For example, the cells may be stably transfected with DNA encoding Cas9. The cells may have DNA encoding Cas9 stably integrated. Expressing the nucleic acid molecule may also be accomplished by integrating the nucleic acid molecule into the genome. U.S. Patent Publication No. 20160186213.

In some embodiments, a Cas enzyme in combination with (and optionally complexed with) a gRNA, a sgRNA, an iRNA, and/or an isgRNA, is delivered to a cell.

The Cas enzyme (e.g., Cas9) may be driven by an inducible promoter (e.g. doxycycline inducible promoter) or a constitutive promoter.

Nucleic acids can be delivered as part of a larger construct, such as a plasmid or viral vector, or directly.

Nucleic acids (DNA or RNA) can be introduced into a population of cells using methods and techniques that are standard in the art, such as infection, transformation, transfection, transduction etc. Non-limiting examples of methods to introduce nucleic acids into cells include lipofectamine transfection, calcium phosphate co-precipitation, electroporation, DEAF-dextran treatment, microinjection, lipid-mediated transfection, viral infection, chemical transformation, electroporation, lipid vesicles, viral transporters, ballistic transformation, pressure induced transformation, viral transduction, particle bombardment, and other methods known in the art.

The nucleic acids may be delivered to cultured cells in vitro. Alternatively, the nucleic acids may be delivered to the cells in a subject. Cells may be isolated from a subject and modified using the present system and method in vitro.

The present disclosure further provides cells produced by the methods described herein, and organisms (such as animals, plants, or fungi) comprising or produced from such cells.

In some embodiments, a population of cells are transiently or non-transiently (e.g., stably) transfected or infected with one or more vectors described herein. In some embodiments, a population of cells are infected or transfected as it naturally occurs in a subject. In some embodiments, a population of cells that are infected or transfected are taken from a subject. In some embodiments, the cells are derived from cells taken from a subject, such as a cell line. Cell lines are available from a variety of sources known to those with skill in the art (see, e.g., the American Type Culture Collection (ATCC)). In some embodiments, a cell infected or transfected with one or more vectors described herein is used to establish a cell line comprising one or more sequences encoding one or more of the present CRISPR components.

Suitable cells include, but are not limited to, mammalian cells (e.g., human cells, mouse cells, rat cells, etc.), primary cells, stem cells, avian cells, plant cells, insect cells, bacterial cells, fungal cells (e.g., yeast cells), and any other type of cells known to those skilled in the art.

The present disclosure also encompasses kits containing the present systems (e.g., the present, constructs, vectors, libraries etc., and/or one or more of the present CRISPR components).

In some embodiments, the kit comprises a vector system and instructions for using the kit. Elements may be provided individually or in combinations, and may be provided in any suitable container, such as a vial, a bottle, or a tube.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form).

The present disclosure encompasses assaying or screening cells expressing the present system (e.g., the present constructs, vectors, libraries etc., and/or one or more of the present CRISPR components).

The ability of an iRNA (or isgRNA) and a gRNA to direct sequence-specific binding of a CRISPR complex to a target sequence may be assessed by any suitable assay, such as by Surveyor assay.

Surveyor assay detects mutations and polymorphisms in a DNA mixture. Surveyor Nuclease can be a member of the CEL family of mismatch-specific nucleases derived from celery. Surveyor Nuclease recognizes and cleaves mismatches due to the presence of single nucleotide polymorphisms (SNPs) or small insertions or deletions. Surveyor nuclease cleaves with high specificity at the 3′ side of any mismatch site in both DNA strands, including all base substitutions and insertion/deletions up to at least 12 nucleotides. Surveyor nuclease technology involves four steps: (i) PCR to amplify target DNA from the cell or tissue samples underwent Cas9 nuclease-mediated cleavage (here we expect to see an nonhomogeneous or mosaic pattern of nuclease treatment on cells, some cells got cuts, some cells don't); (ii) hybridization to form heteroduplexes between affected and unaffected DNA (Because the affected DNA sequence will be different from the affected, a bulge structure resulted from the mismatch can form after denature and rename): (iii) treatment of annealed DNA with Surveyor nuclease to cleave heteroduplexes (cut the bulges); and (iv) analysis of digested DNA products using the detection/separation platform of choice, for instance, agarose gel electrophoresis. The Cas9 nuclease-mediated cleavage efficacy can be estimated by the ratio of Surveyor nuclease-digested over undigested DNA. Surveyor mutation assay kits are commercially available from integrated DNA Technologies (IDT), Coraville, Iowa.

Similarly, cleavage of a target sequence may be evaluated in a test tube by providing the target sequence, components ala CRISPR complex, including the iRNA to be tested, gRNA to be tested, and a control iRNA (or isgRNA) different from the test iRNA (or isgRNA) sequence, and/or a control gRNA different from the test gRNA sequence, and comparing binding or rate of cleavage at the target sequence between the test and control reactions. Other suitable assays are also possible.

To determine the function of the genes modulated by the present CRISPR-Cas system, cells contacted with the present system are compared to control cells, e.g., without the CRISPR-Cas system or with a non-specific CRISPR-Cas system, to examine the extent of modification (e.g., inhibition or activation) of gene activity, and/or change (e.g., increase or decrease) in gene expression level. Control samples may be assigned a relative gene expression value of 100%. The present CRISPR-Cas system may decrease or increase gene activity and/or gene expression level by about or at least about 80%, 50%, 25%, 10%, 5%, 2-fold, 5-fold, 10-fold, 20-fold, at least about 1.2 fold, at least about 1,4 fold, at least about 1.5 fold, at least about 1.8 fold, at least about 2 fold, at least about 3 fold, at least about 4 fold, at least about 5 fold, at least about 6 fold, at least about 7 fold, at least about 8 fold, at least about 10 fold, at least about 15 fold, at least about. 20 fold, at least about 25 fold, at least about 30 fold, at least about 35 fold, at least about 40 fold, at least about 50 fold, at least about 60 fold, at least about 70 fold, at least about 80 fold, at least about 90 fold, at least about 100 fold, at least about 200 fold, at least about 250 fold, at least about 300 fold, at least about 400 fold, or at least about 500 fold, compared to the gene expression level and/or gene activity in the control.

The expression level of the modified gene may be at least about 1.2 fold, at least about 1.4 fold, at least about 1.5 fold, at least about 1.8 fold, at least about 2 fold, at least about 3 fold, at least about 4 fold, at least about 5 fold, at least about 6 fold, at least about 7 fold, at least about. 8 fold, at least about 10 fold, at least about 15 fold, at least about 20 fold, at least about 25 fold, at least about 30 fold, at least about 35 fold, at least about 40 fold, at least about 50 fold, at least about 60 fold, at least about 70 fold, at least about 80 fold, at least about 90 fold, at least about 100 fold, at least about 200 fold, of the expression level of the gene in its natural form (e.g., in control cells).

For example, an assay is used to determine whether or not the gene targeting is associated with a selected phenotype. It can be determined whether two or more genes are associated with the same phenotype. The present system (e.g., libraries, constructs, vectors) can also be used to determine whether a gene participates with other genes in a particular phenotype.

A phenotype refers to any phenotype, e.g., any observable characteristic or functional effect that can be measured in an assay such as changes in cell growth, proliferation, morphology, enzyme function, signal transduction, expression patterns, downstream expression patterns, reporter gene activation, hormone release, growth factor release, neurotransmitter release, ligand binding, apoptosis, and product formation. A candidate gene is “associated with” a selected phenotype if modulation of gene expression of the candidate gene causes a change in the selected phenotype.

In certain embodiments, gene expression and/or modification can be assayed by determining any parameter that is indirectly or directly affected by the expression of the target genes. Such parameters include, e.g., changes in RNA or protein levels, changes in RNA stability, changes in protein activity, changes in product levels, changes in downstream gene expression, changes in reporter gene transcription or expression (e.g., via chemiluminescence, fluorescence, calorimetric reactions, antibody binding, inducible markers, ligand binding assays, such as assaying luciferase, CAT, beta-galactosidase, beta-glucuronidase, GFP (see, e.g., Mistili & Spector, Nature Biotechnology 15:961-964 (1997)); changes in signal transduction, changes in phosphorylation anchor dephosphorylation, changes in receptor-ligand interactions, changes in second messenger (such as cGMP and inositol triphosphate (IP3)) concentrations, changes in cell growth, changes in intracellular calcium levels; changes in cytokine release, and changes in neovascularization, etc., as described herein. These assays can be in vitro, in vivo, and ex vivo.

Such assays include, e.g., transformation assays, e.g., changes in proliferation, anchorage dependence, growth factor dependence, foci formation, growth in soft agar, tumor proliferation in nude mice, and tumor vascularization in nude mice; apoptosis assays, e.g., DNA laddering and cell death, expression of genes involved in apoptosis; signal transduction assays, e.g., changes in intracellular calcium, cAMP, cGMP, IP3, changes in hormone and neurotransmitter release; receptor assays, e.g., estrogen receptor and cell growth; growth factor assays, e.g., EPO, hypoxia and erythrocyte colony forming units assays; enzyme product assays, e.g., FAD-2 induced oil desaturation; transcription assays, e.g., reporter gene assays; and protein production assays, e.g., VEGF ELISAs.

The present functional screens allow for discovery of novel human and mammalian therapeutic applications, including the discovery of novel drugs, for, e.g., treatment of genetic diseases, cancer, fungal, protozoal, bacterial, and viral infection, ischemic, vascular disease, arthritis, immunological disorders, etc.

In some embodiments, cells transiently or non-transiently transfected or infected with one or more vectors described herein, or cell lines derived from such cells are used in assessing one or more test compounds.

The present methods and systems may be used for mapping genetic interactions, large-scale phenotyping, gene-to-function mapping, meta-genomic analysis, drug screening, disease diagnosis, prognosis, etc. WO2015071474.

The present methods and systems may be used to treat a genetic disorder, including genetic disorders with one or more insertions, deletions, and/or point mutations (e.g. Duchenne muscular dystrophy).

The present methods and systems may be used to develop personalized gene therapy strategies for subjects with genetic mutations.

The present methods and vectors can be used to identify two or more inhibitors targeting two or more genes. The inhibitors can be used to treat disorders or diseases.

For example, the inhibitors identified by the present method may be used to reduce or inhibit cell proliferation. The cell may be a cancer cell. The inhibitors may be used to treat cancer. In some embodiments, the inhibitors are a sequence encoding an iRNA, a sequence encoding an isgRNA, a sequence encoding a gRNA; an antisense RNA, an snRNA or shRNA; and/or a small molecule.

The present application provides methods for treating a disorder (e.g., cancer, or other disorders described herein) in a subject comprising administering to the subject a combination of two or more inhibitors targeting two or more genes. The inhibitors are administered in a therapeutically effective amount.

In some embodiments, the effective amount of each of the two or more inhibitors administered in the combination is less than the effective amount of the inhibitor when not administered in the combination.

The present methods and systems may be used for CRISPR display which is a targeted localization method that uses Sp. Cas9 to deploy large RNA cargos to DNA loci. For example, one or more RNA domains may be inserted into one or more gRNAs. In some embodiments, the vector encodes a gRNA fused to one or more RNA domain. In some embodiments, the RNA is a non-coding RNA or fragment thereof. In such embodiments, the RNA domain may be targeted to a DNA loci. Shechner et al., CRISPR Display: a modular method for locus-specific targeting of long noncoding RNAs and synthetic RNA devices in vivo, Nature Methods, 2015, 12(7):664-670.

The present systems may be analyzed by sequencing or by microarray analysis. It should be appreciated that any means of determining DNA sequence is compatible with identifying one or more DNA elements.

The DNA may be extracted and sequenced to identify a sequence encoding an iRNA, a sequence encoding an isgRNA, and/or a sequence encoding a gRNA, and/or genetic modifications.

DNA may be amplified via polymerase chain reaction (PCR) before being sequenced.

The DNA may be sequenced using vector-based primers; or a specific gene is sought by using specific primers. PCR and sequencing techniques are well known in the art; reagents and equipment are readily available commercially.

Non-limiting examples of sequencing methods include Sanger sequencing or chain termination sequencing, Maxam-Gilbert sequencing, capillary array DNA sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nat. Biotechnol., 16:381-384 (1998)), and sequencing by hybridization (Chee, et al., Science, 274:610-614 (1996); Drmanac et al., Science, 260:1649-1652 (1993); Drmanac et al., Nat. Biotechnol., 16:54-58 (1998)), NGS (next-generation sequencing) (Chen et al., Genome Res. 18:1143-1149 (2008); Srivatsan et al. PloS Genet, 4:e1000139 (2008)), Polony sequencing (Porreca et al., Curr. Protoc. Mol. Biol. Chp. 7; 7.8 (2006), ion semiconductor sequencing (Elliott et al., J. Biomol Tech. 1:24-30 (2010), DNA nanoball sequencing (Kaji et al., Chem Soc Rev 39:948-56 (2010), single-molecule real-time sequencing (Flusberg et al., Nat. Methods 6:461-5 (2010), sequencing by synthesis (e.g., Illumina/Solexa sequencing), sequencing by ligation, sequencing by hybridization, nanopore DNA sequencing (Wanunu, Phys Life Rev 9:125-58 (2012), massively Parallel Signature Sequencing (MASS); pyro sequencing, SOLID sequencing (McKeman et al. 2009 Genome Res 19:1527-1541; Shearer et al. 2010 Proc Natl Acad Sci USA 107:21104-21109); shortgun sequencing; Heliscope single molecule sequencing; single molecule real time (SMRT) sequencing. U.S. Patent Publication No. 20140329705.

High-throughput sequencing, next-generation sequencing (NGS), and/or deep-sequencing technologies include, but are not limited to, Illumina/Solex sequencing technology (Bentley et al. 2008 Nature 456:53-59), Roche/454 (Margulies et al. 2005 Nature 437:376-380), Pacbio, (Flusberg et al. 2010 Nature methods 7:461-465; Korlach et al. 2010 Methods in enzymology 472:431-455; Schadt et al. 2010 Nature reviews. Genetics 11:647-657; Schadt et at 2010 Human molecular genetics 19:R227-240; Eid et al. 2009 Science 323:133-138; Imelfort and Edwards, 2009 Briefings in bioinformatics 10:609-618), Ion Torrent (Rothberg et al. 2011 Nature 475:348-352)) and more. For example, Polony technology utilizes a single step to generate billions of “distinct clones” for sequencing. As another example, ion-sensitive field-effect transistor (ISFET) sequencing technology provides a non-optically based sequencing technique. U.S. Patent Publication No. 20140329712.

Several methods of DNA extraction and analysis are encompassed in the present disclosure. As used herein “deep sequencing” indicates that the depth of the process is many times larger than the length of the sequence under study. Deep sequencing is encompassed in next generation sequencing methods which include but are not limited to single molecule realtime sequencing (Pacific Bio), Ion semiconductor (Ion torrent sequencing), Pyrosequencmg (454), Sequencing by synthesis (lilumina), Sequencing by ligations (SOLID sequencing) and Chain termination (Sanger sequencing).

Sequencing of the DNA after introduction of the present system into cells can identify the specific genes (e.g., the specific pair(s) of genes) affected by the CRISPR system corresponding to a selected phenotype.

Sequencing reads may be first subjected to quality control to identify overrepresented sequences and low-quality ends. The start and/or end of a read may or may not be trimmed. Sequences mapping to the genome may be removed and excluded from further analysis. As used herein, the term “read” refers to the sequence of a DNA fragment obtained after sequencing. In certain embodiments, the reads are paired-end reads, where the DNA fragment is sequenced from both ends of the molecule.

The present systems and methods may be used to manipulate nucleic acids of a suitable organism. The organism may be a eukaryotic organism, including human and non-human eukaryotic organisms. The organism may be a multicellular eukaryotic organism. The organism may be an animal, for example a mammal such as a mouse, rat, or rabbit. Also, the organism may be an arthropod such as an insect. The organism also may be a plant or a fungus. The organism may be prokaryotic.

In one embodiment, the cell is a mammalian cell, such as a human cell. Human cells may include human embryonic kidney cells (e.g., HEK293T cells), human dermal fibroblasts, human cancer cells, etc. The organism may be a mammal, such as humans, dogs and cats, farm animals such as cows, pigs, sheep, horses, goats and the like, and laboratory animals (e.g., rats, mice, guinea pigs, and the like). The present system may be delivered by plasmids or delivered by viruses such as lentiviruses, adenoviruses or AAVs.

In another embodiment, the cell is a yeast cell. The organism may be a yeast. The present system may be delivered by plasmids or shuttle vectors. In yet another embodiment, the cell is a bacterial cell. The organism may be bacteria. The present system may be delivered by plasmids or phages.

The following are examples of the present invention and are not to be construed as limiting.

### Example 1

The adaptive bacterial immunity system, CRISPR/Cas9, has provided researchers with an effective tool to edit the DNA sequences (1). It paved the way for directing the Cas9 nuclease to target a particular DNA sequence by just replacing the base-pairing sequences of the sgRNA. Additionally, the ability to localize the enzyme within the nucleotide prevision allowed us to monitor the cellular reactions involving DNA and RNA by using nuclease deficient Cas9 (2). However, the requirement of a Protospacer Adjacent Motif (PAM) sequence to be present immediately adjacent to a target region limits the possible genomic sequences that are suitable for Cas9 targeting. Furthermore, the PAM requirement also increases the likelihood of off-target mutations on other chromosomes (3).

Sternberg et al. observed cleavage of single-stranded DNAs without PAM. Ma et al. also reported that CRISPR/Cas9 system can cleave single-stranded DNAs in the absence of the PAM region (4, 5). Therefore, unlike the dsDNA substrates, it is possible to cleave ssDNAs with or without PAM motifs.

Here a novel strategy of cleaving any dsDNA target independent of PAM is disclosed. Specifically, the two strands of the dsDNA are transiently separated by using an invader RNA (iRNA). Once the strands are separated and a bulge is fanned, the Cas9 complex binds to the target sequence of the target strand and cleave the DNA. In the absence of the timely strand separation induced by the iRNA, Cas9 complexes would skip the target due to the lack of PAM sequences (4).

The single guide RNA structure may be extended to include an invader RNA which can hybridize with (or is complementary to) a sequence in the non-target strand of the double-stranded DNA, to initiate strand separation, followed by Cas9 binding, and the cleavage of the target, as shown in FIG. 1A. Once the first cleavage occurs on the initial target strand, the complementary non-target strand may also be targeted with another Cas9 complex. This strategy eliminates the requirement for PAM.

Another strategy to separate the strands of a dsDNA includes using an invader RNA (iRNA) that is separate from the guide RNA, crRNA and/or tracrRNA as shown in FIG. 1B.

To test the PAM-independent DNA cleavage strategy, two target sequences within a 2148-bp dsDNA (SEQ ID NO: 1) were selected. The two target sequences were adjacent to ACG or TAT sequences. The selected target sequences are not near any of the known PAM sequences (6). The 2148-bp dsDNA was targeted by either (i) Cas9 plus an isgRNA, or (ii) Cas9 plus an sgRNA and iRNA.

The two isgRNAs were named as isgRNA 580 (transcribed by the DNA template as specified in SEQ ID NO: 6) and isgRNA 591 (transcribed by the DNA template as specified in SEQ ID NO: 7) based on their respective cleavage sites in the dsDNA. IsgRNA 580 and isgRNA 591 target different strands of the dsDNA. IsgRNA 580 guides DNA cleavage at nucleotide 580 of one strand of the dsDNA. IsgRNA 591 guides DNA cleavage at nucleotide 591 of the other strand of the dsDNA. Each of isgRNA 580 and isgRNA 591 contains an iRNA segment (iRNA 580 and iRNA 591, respectively) that can hybridize to the non-target DNA strand.

Similarly, the two sgRNAs were named as sgRNA 580 (transcribed by the DNA template as specified in SEQ ID NO: 10) and sgRNA 591 (transcribed by the DNA template as specified in SEQ ID NO: 11) based on their respective cleavage sites in the dsDNA. SgRNA 580 and sgRNA 591 target different strands of the dsDNA. SgRNA 580 guides DNA cleavage at nucleotide 580 of one strand of the dsDNA. SgRNA 591 guides DNA cleavage at nucleotide 591 of the other strand of the dsDNA. Each of sgRNA 580 and sgRNA 591 was used in combination with an iRNA that can hybridize to the non-target DNA strand. SgRNA 580 was used in combination with iRNA 580 (SEQ ID NO: 8). SgRNA 591 was used in combination with iRNA 591 (SEQ ID NO: 9). The molar ratio of the iRNA to the sgRNA was 10:1 or 100:1.

The reaction buffer was 20 mM HEPES, 100 mM NaCl, 5 mM MgCl₂, 0.1 mM EDTA, pH 6.5. The reactions were incubated overnight at room temperature (ambient temperature).

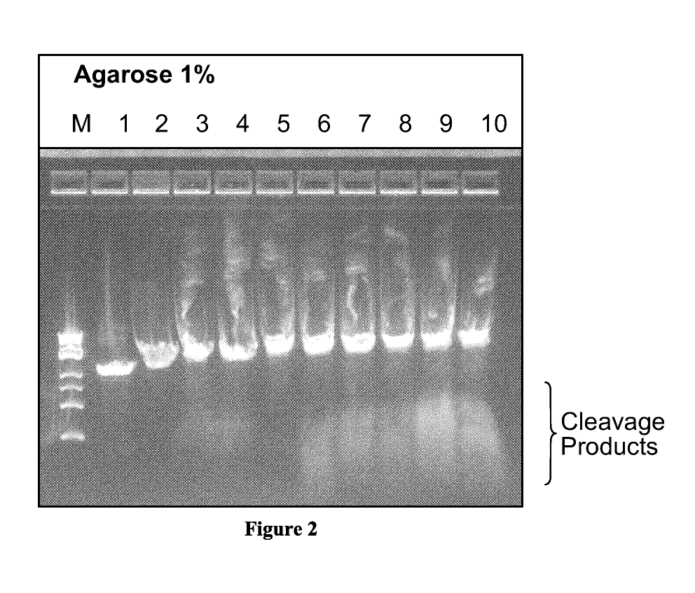

The PAM-free DNA cleavage reaction and the control samples (including dsDNA only, Cas9 without gRNA, and Cas9 with wild type gRNA in the absence of iRNA) were analyzed with gel electrophoresis (FIG. 2). The iRNA has a total length of 28 nt which includes a 20-nt sequence that is complementary to (can hybridize with) the target strand of the 2.1-kb dsDNA and a 4-nt sequence on each of the 5′-end and 3′-end of the 20-nt sequence. The isgRNA has a total length of 134 nt which includes the same iRNA sequence linked to the sgRNA by at least seven uracil nucleobases UUUUUUU. The crRNA and tracrRNA are wild-type and have a length of 36 nt and 67 nt.

FIG. 2 shows that dsDNA can be cleaved in the absence of PAM by two different approaches involving an invader RNA. See, FIG. 2, lanes 3-4: Cas9 and isgRNA (sgRNA covalently linked with invader RNA); and lanes 6-10: Cas9, sgRNA (crRNA-tracrRNA), and a separate invader RNA. Specifically, Lane 3: 8 nM dsDNA, 80 nM Cas9, and 80 nM isgRNA 591 (sgRNA covalently linked to invader RNA). The molar ratios of DNA Cas9:isgRNA 591 are 1:10:10. Cleaved DNA can be seen. Lane 4: 8 nM dsDNA, 80 nM Cas9, and 80 nM isgRNA 580 (sgRNA covalently linked to invader RNA). The molar ratios of DNA:Cas9:isgRNA 580 are 1:10:10. Cleaved DNA can be seen. Lane 6: molar ratios of DNA:Cas9:sgRNA 591: IRNA 591 are 1:10:10:100. Lane 6 includes 8 nM dsDNA, 80 nM Cas9, 80 nM sgRNA. 591 and 800 nM IRNA 591. Lane 7: molar ratios of DNA:Cas9:sgRNA 580:iRNA 580 are 1:10:10:100. Lane 7 includes 8 nM dsDNA, 80 nM Cas9, 80 nM sgRNA 580 and 800 nM iRNA 580. Lane 8: molar ratios of DNA:Cas9:sgRNA 591:iRNA 591 are 1:10:10:1000. Lane 8 includes 8 nM dsDNA, 80 nM Cas9, 80 sgRNA 591 and 8 μM iRNA 591. Lane 9: molar ratios of DNA:Cas9:sgRNA580:iRNA 580 are 1:10:10:1000. Lane 9 includes 8 nM dsDNA, 80 nM Cas9, 80 nM sgRNA 580 and 8 μM iRNA 580. Lane 10: molar ratios of DNA:Cas9:sgRNA:iRNA 580:Cas9:sgRNA 591:iRNA 591 are 1 DNA:10 Cas9:10 sgRNA 580:100 iRNA 580:10 Cas9:10 sgRNA 591:100 iRNA 591. For the reaction of lane 10, the set of Cas9, sgRNA 591 and iRNA 591 was added 30 minutes later than the set of Cas9, sgRNA 580 and iRNA 580. Lane 10 includes 8 nm dsDNA, 80 nM Cas9, 80 nM sgRNA 580, 800 nM iRNA 580, 80 nM Cas9, 80 nM sgRNA 591, and 800 nM iRNA 591. Controls include lane 1 (dsDNA only), lane 2 (Cas9 without gRNA), and lane 3 (Cas9 with sgRNA). 1 kb DNA ladder (NEB) was used for lane M. Lane 5: 8 nM dsDNA, 80 nM Cas9 and 80 nM sgRNA. For lane 5, the molar ratios of DNA:Cas9:sgRNA are 1:10:10.

The data demonstrate that the DNA cleavages are facilitated by an invader RNA when supplied either separately from, or covalently linked to, the gRNA. Relatively broad distribution of some cleaved DNA bands may indicate that the cleavages occur within the target sequence but not at the same nucleotides. This finding also suggests the possibility of creating DNA breaks with sticky ends by shifting the target sequences of the DNA strands.

To study the PAM-independent DNA cleavage, different sizes of bulged DNA will be investigated regarding their efficiencies by using the DNA sequencing technologies. Changing the length of the invader RNA alters the lifetime of the invader RNA-DNA complex which may affect Cas9 binding on the target DNA strand. Also, employing mismatches on the invader RNA with the recently engineered Cas9 enzymes which have variant PAM recognition mutations (7) will help reveal more molecular details of DNA scission in the absence of PAM. Additionally, different strategies to link an invader RNA and a guide RNA will be examined, and the size of the linker will be varied.

Gene editing using the present systems and methods in eukaryotic cells will also be investigated. Several RNA delivery strategies from the RNA interference system may be adopted to introduce the RNAs (iRNA, isgRNA, sgRNA, crRNA and/or tracrRNA) into the target cells.

### Example 2

PAM-independent DNA cleavage by Cas9 was studied by using either the isgRNA or the separate iRNA approaches.

### Methods

Two isgRNAs were generated by linking the sgRNA with the corresponding 28-nt iRNA (complementary to the non-target DNA strand) by at least seven uracil nucleobases UUUUUUU. The two isgRNAs were named as isgRNA 580 (transcribed by the DNA template as specified in SEQ ID NO: 6) and isgRNA 591 (transcribed by the DNA template as specified in SEQ ID NO: 7) based on their respective cleavage sites in the dsDNA. IsgRNA 580 and isgRNA 591 target different strands of the dsDNA. IsgRNA 580 guides DNA cleavage at nucleotide 580 of one strand of the dsDNA. IsgRNA 591 guides DNA cleavage at nucleotide 591 of the other strand of the dsDNA. Each of isgRNA 580 and isgRNA 591 contains an iRNA segment that can hybridize to the non-target DNA strand.

Similarly, the two sgRNAs were named as sgRNA 580 (transcribed by the DNA template as specified in SEQ ID NO: 10) and sgRNA 591 (transcribed by the DNA template as specified in SEQ ID NO: 11) based on their respective cleavage sites in the dsDNA. SgRNA 580 and sgRNA 591 target different strands of the dsDNA. SgRNA 580 guides DNA cleavage at nucleotide 580 of one strand of the dsDNA. SgRNA 591 guides DNA cleavage at nucleotide 591 of the other strand of the dsDNA. Each of sgRNA 580 and sgRNA 591 was used in combination with an IRNA that can hybridize to the non-tan et DNA strand. SgRNA 580 was used in combination with iRNA 580 (SEQ ID NO: 8). SgRNA 591 was used in combination with iRNA 591 (SEQ ID NO: 9).

A 2148-bp dsDNA substrate (SEQ ID NO: 1) was targeted by either (i) Cas9 plus an isgRNA or (ii) Cas9 plus an sgRNA and an iRNA. The target regions do not contain canonical PAM sequences.

To prepare the cleavage assays, Cas9 molecules were incubated with the guide RNA (sgRNA or isgRNA) for 10 mins in the Cas9 cleavage buffer. Then dsDNA was introduced to the assay. For the reactions with sgRNA and iRNA, 10× more separate iRNA molecules were added to the assays containing the sgRNA. The molar ratios of dsDNA:Cas9:sgRNA:iRNA are 1:10:10:100. The molar ratio of the iRNA to the sgRNA was 10:1). The molar ratios of dsDNA Cas9 isgRNA were 1:10:10. The concentrations of the various components were as follows (in reactions where they were present): 30 nM dsDNA, 300 nM sgRNA, 300 nM isgRNA, and 3 μM iRNA.

The cleavage assays were incubated overnight at room temperature. Finally, they were loaded to the 1% agarose gel and imaged for the observation of the cleavage bands.

### Results

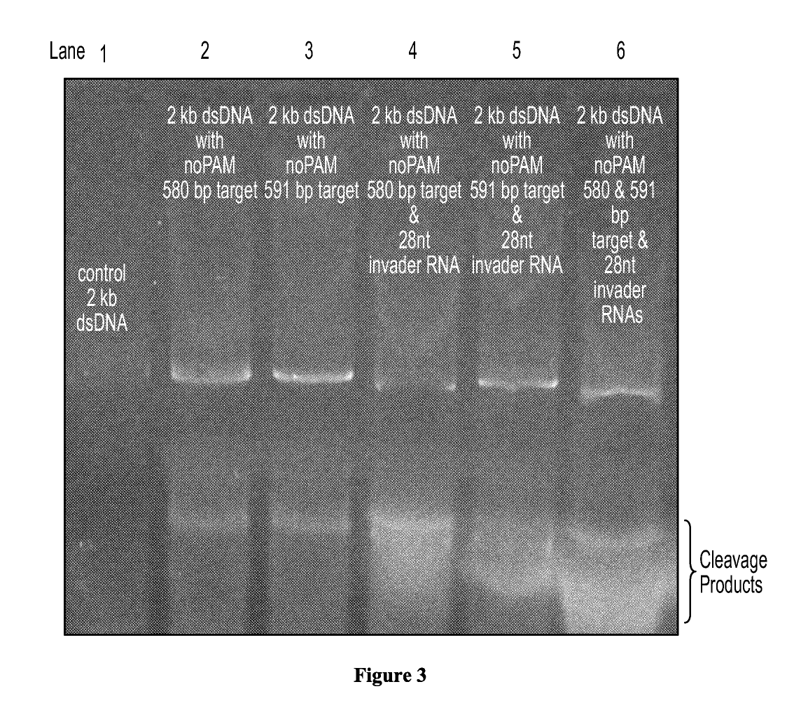

As a control experiment, only dsDNA was loaded on lane 1 (FIG. 3). When dsDNA was attacked by Cas9/isgRNA, a longer (around 1.5 kb) cleavage fragment was observed. However, a second short fragment was not observed. This could indicate that only one strand of the dsDNA was cleaved when attacked by a single isgRNA as observed in lanes 2 and 3 (FIG. 3). Interestingly, when only one strand of dsDNA was attacked by regular Cas9/sgRNA and supplied with a separate iRNA, two cleavage fragments were observed in lanes 4 and 5 (FIG. 3). Finally, a higher cleavage efficiency was observed when both strands of dsDNA were targeted with two separate sgRNA and iRNA in lane 6 (FIG. 3).

### Example 3

When studying ssDNA cleavage by the Cas9 systems, Ma et al. only tested the cleavage activity with a crRNA in the absence of any tracrRNA. Ma et al., Single-Stranded DNA Cleavage by Divergent CRISPR-Cas9 Enzymes, Mol. Cell, 2015, 60 (3), p398-407. Here, a cleavage assay of native guide RNA system (including both a crRNA and a tracrRNA) coupled with an invader was investigated.

### Method

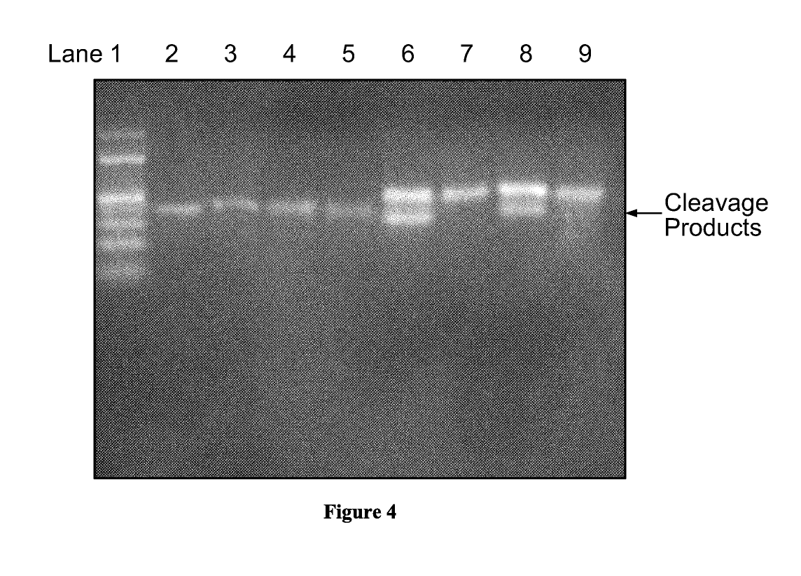

Two 80-nt ssDNA substrates, ssDNA-1 (SEQ ID NO: 2) and ssDNA-2 (SEQ ID NO: 3) were from IDT. Four different assays were prepared with the two ssDNAs. The assays had (i) the ssDNA only (lanes 2 and 3 of FIG. 4); (ii) the ssDNA and Cas9 without any guide RNA (lanes 4 and 5 of FIG. 4); (iii) the ssDNA, Cas9 and crRNA-tracrRNA and a 28-nt iRNA which complements with a sequence of the ssDNA (lanes 6 and 8 of FIG. 4); or (iv) ssDNA and iRNA (lanes 7 and 9 of FIG. 4).

All of the assays were mixed in the Cas9 cleavage buffer (NEB) and incubated for 2 hours at room temperature. The molar ratios of ssDNA:Cas9:sgRNA:iRNA are 1:10:10:100. The mixtures were loaded to a 4% agarose gel which then was stained with Gelred (Biotium).

### Results

The cleavage activity was observed when an iRNA was present (lanes 6 and 8 of FIG. 4). The iRNA hybridizes with ssDNA in the absence of PAM and creates a bulge, before Cas9 cleaves the ssDNA (lanes 6 and 8, lower band). The upper hands in lanes 6 and 8 correspond to the ssDNA associated with the iRNA (same as lanes 7 and 9).

### Example 4

### Cas9 and Guide RNA Preparation

Wild-type Cas9 enzymes from S. pyogenes will be obtained from New England BioLabs Inc. (M0386S, Ipswich, Mass., USA). The invader single guide RNA (isgRNA) will be designed by using the two loops described at Jinek et al. and extending it from 3′ end as it can also hybridize with the non-target DNA strand. Jinek et al., A Programmable Dual-RNA-Guided DNA Endonuclease in Adaptive. Bacterial Immunity, Science, 2012, 337: 816. After an ssDNA break occurs independent of the PAM sequence on one strand of the target DNA, another isgRNA is used similarly to target the complementary strand of the dsDNA. Thus, a double-strand break can be achieved.

To transcribe the isgRNA, a single-stranded DNA template will be purchased from Integrated DNA Technologies (IDT, Coralville, Iowa, USA). The complementary strand will be generated by polymerase chain reaction (PCR) using high fidelity Phusion DNA polymerase (M0530S, New England BioLabs Inc., Ipswich, Mass., USA). Thus, a double-stranded DNA encoding the isgRNA will be produced.

The formation of dsDNA will be confirmed using gel electrophoresis. In vitro transcription of isgRNA will be performed with the T7 RNA polymerase (ThermoFisher Scientific, Waltham, Mass., USA) by using PCR generated dsDNA template which consists the T7 promoter sequence. After transcription, isgRNA will be purified using Zymo RNA Clean & Concentrator kit (Zymo Research, Irvine, Calif., USA). The nuclease activity of the Cas9 enzymes and the transcription of the isgRNA will be tested with the cleavage assay for the 3.6 kb plasmid DNA which includes the target sequence and PAM (pT7CFE1TNHis, ThermoFisher Scientific, Waltham, Mass., USA). Linear dsDNA product will be observed with the electrophoresis gel assays.

### In Vivo Cas9 Reactions

The activity of the Cas9 enzyme in the absence of PAM will be tested by studying expression level of the GFP gene (enhancing or silencing) in the CHO-K1 cells. For silencing the GFP gene, CHO-K1 cells will be transfected with CRISPR nuclease mRNA and in vitro transcribed isgRNA. Cultured transfected cells will be assessed by fluorescence microscopy for the GFP signal.

For CRISPR activator assays, dCas9 mRNA and the in vitro transcribed isgRNA will be coupled with MS2 loop and the upregulation will be observed by comparing GFP signal with the control cells.

### In Vitro Cas9 Reactions

Cas9 reactions will be prepared according to the NEB product manual. For the 30 μL of reaction volume, 6 μL of 5× DP buffer will be added to 12 μL of nanopure water. Then 9 μL of 300 nM isgRNA and 3 μM stock Cas9 nuclease added to the solution. The reaction mixture will be incubated at 22 C for at least 15 minutes. Afterwards, for bulk experiments, Cas9-isgRNA complexes mixed with target DNA samples or for AFM experiments, 1 μL of reaction volume will be gently added to the mica without touching the surface.

Cleavage of ssDNA will also be tested using an sgRNA and an invader RNA. Additionally, excessive amount of 28-34 nt invader RNA will be mixed with target DNA to create a bulge on the target site without a PAM region. Then, single strand breaks will be detected by PCR ructions and gel electrophoresis assays.

### Atomic Force Microscopy (AFM)

The mechanism and dynamics of the present system may be investigated using AFM.

Poly-L-ornithine mediated surface will be used to immobilize the DNA. A stock solution of 0.1 mg mL-1 poly-L-ornithine (Sigma, St. Louis, Mo., USA) will be prepared. 20 μL of the droplet will be applied for 1 minutes to a freshly cleaved mica surface (VWR, Radnor, Pa., USA). Then, the mica surface will be held with 500 μL nanopure water and dried with N₂ gas. Afterwards, a drop of 30 μL solution containing 0.5 ng μL⁻¹ template DNA in the deposition buffer (4 mM HEPES, 1 mM KCl, 1 mM MgCl2 pH 7.0 at 22 Celsius) will be applied to mica surface for 5 minutes. Then, the surface will he rinsed with 400 μL deposition buffer (DP). Finally, 20 μL of DP buffer applied to the surface to keep the surface wet.

Atomic Force Microscopy will be performed in liquid with tapping mode using commercial AFM microscopes (Bruker Multimode V and Bioscope II) with either AC40 probes (Bruker, Billerica, Mass., USA) with 0.09 N/m nominal spring constant with 25 kHz resonant frequency or HYDRA probes (Applied NanoStructures, Mountain View, Calif. USA) with 0.284 N/m nominal spring constant with 66 kHz resonant frequency at 22 C temperature. In general, 512×256 pixel images over 2-micron square areas acquired with 1 Hz scan rate. When a particular DNA-Cas9 interaction will be investigated, usually an area of 664 nm square will be scanned up to 4 Hz rate.

Acquired images will be flattened by the Nanoscope software (Bruker, Billerica, Mass., USA) by using third order polynomial. Then contour length of the DNA will be transformed to ImageJ and traced with NeuronJ package. After the nuclease event, the long and short piece of the DNA will be also traced with the same method to confirm the specificity of the target site. Some images will be processed with SPIP (Image Metrology, Hørsholm, Denmark).

### REFERENCES

1) Jinek, Martin, Krzysztof Chylinski, Ines Fonfara, Michael Hauer, Jennifer A. Doudna, and Emmanuelle Charpentier, “A programmable dual-RNA-guided DNA endonuclease in adaptive bacterial immunity.” Science 337, no. 6096 (2012): 816-821.

2) Nelles, D. A., Fang, M. Y., O'Connell, M. R., Xu, J. L., Markmiller, S. J., Doudna, J. A., & Yeo, G. W. (2016). Programmable RNA tracking in live cells with CRISPR/Cas9. Cell, 165(2), 488-49

3) Kuscu, Cem, Sevki Arslan, Ritambhara Singh, Jeremy Thorpe, and Mazhar Adli. “Genome-wide analysis reveals characteristics of off-target sites bound by the Cas9 endonuclease.” Nature biotechnology 32, no. 7 (2014): 677-683.

4) Sternberg, Samuel H., Sy Redding, Martin Jinek, Eric C. Greene, and Jennifer A. Doudna. “DNA interrogation by the CRISPR RNA-guided endonuclease Cas9.” Nature 507, no. 7490 (2014): 62-67.

5) Ma, Enbo, Lucas B. Harrington, Mitchell R. O'Connell, Kaihong Zhou, and Jennifer A. Doudna. “Single-stranded DNA cleavage by divergent CRISPR-Cas9 enzymes.” Molecular cell 60, no. 3 (2015): 398-407.

6) Leenay, Ryan T., Kenneth R. Maksimchuk, Rebecca A. Slotkowski, Roma N. Agrawal, Ahmed A. Gomaa, Alexandra E. Briner, Rodolphe Barrangou, and Chase L. Beisel. “Identifying and visualizing functional PAM diversity across CRISPR-Cas systems.” Molecular cell 62, no. 1 (2016): 137-147.

7) Kleinstiver, Benjamin P., Michelle S. Prew, Shengdar Q. Tsai, Ved V. Topkar, Nhu T. Nguyen, Zongli Zheng, Andrew P W Gonzales et al. “Engineered CRISPR-Cas9 nucleases with altered PAM specificities.” Nature 523, no. 7561 (2015): 481-485.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

What is claimed is:

1. A system that targets a target sequence in a double-stranded DNA, the system comprising:
(i) a first RNA comprising:
(a) a first segment that hybridizes with the target sequence in a target strand of the double-stranded DNA; and
(b) a second segment that hybridizes with the first segment to form a double-stranded protein-binding motif;
(ii) a second RNA that hybridizes with a sequence in a non-target strand of the double-stranded DNA; and
(iii) a Cas enzyme or a variant thereof,
wherein the first RNA forms a complex with the Cas enzyme or a variant thereof.

2. The system of claim 1, wherein the second RNA has about 14 to about 34 nucleotides.

3. The system of claim 1, wherein the target sequence is not immediately flanked by a protospacer adjacent motif (PAM).

4. The system of claim 1, wherein the target sequence is immediately flanked by a protospacer adjacent motif (PAM).

5. The system of claim 1, wherein the Cas enzyme is Cas9, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1 Csx15, Csf1, Csf2, Csf3, Csf4, Cpf1, homologs thereof, orthologs thereof, or modified versions thereof.

6. The system of claim 1, wherein the Cas enzyme is Cas9.

7. The system of claim 1, wherein the Cas enzyme comprises one or more mutations.

8. The system of claim 1, wherein the Cas enzyme is codon-optimized for expression in a eukaryotic cell.

9. The system of claim 8, wherein the eukaryotic cell is a mammalian or human cell.

10. The system of claim 1, wherein the Cas enzyme or a variant thereof cleaves the target sequence.

11. A DNA-targeting RNA, comprising:
(i) a first segment that hybridizes with a target sequence in a target strand of a double-stranded DNA;
(ii) a second segment that hybridizes with the first segment to form a double-stranded protein-binding motif; and
(iii) a third segment that hybridizes with a sequence in a non-target strand of the double-stranded DNA,
wherein the RNA forms a complex with a Cas enzyme or a variant thereof.

12. The RNA of claim 11, wherein the third segment has about 14 to about 34 nucleotides.

13. The RNA of claim 11, wherein the target sequence is not immediately flanked by a protospacer adjacent motif (PAM).

14. The RNA of claim 11, wherein the target sequence is immediately flanked by a protospacer adjacent motif (PAM).

15. The RNA of claim 11, wherein the Cas enzyme is Cas9, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, orthologs thereof, or modified versions thereof.

16. The RNA of claim 11, wherein the Cas enzyme is Cas9.

17. The RNA of claim 11, wherein the Cas enzyme or a variant thereof cleaves the target sequence.

18. A DNA polynucleotide encoding the RNA of claim 11.

19. A vector comprising the DNA polynucleotide of claim 18.

20. A cell comprising the DNA polynucleotide of claim 18.

21. A system that targets a target sequence in a double-stranded DNA, the system comprising:
(i) a first RNA that hybridizes with the target sequence in a target strand of the double-stranded DNA and
(ii) a second RNA that hybridizes with the first RNA to form a double-stranded protein-binding motif;
(iii) a third RNA that hybridizes with a sequence in a non-target strand of the double-stranded DNA; and
(iv) a Cas enzyme or a variant thereof.

22. The system of claim 21, wherein the third RNA has about 14 to about 34 nucleotides.

23. The system of claim 21, wherein the target sequence is not immediately flanked by a protospacer adjacent motif (PAM).

24. The system of claim 21, wherein the target sequence is immediately flanked by a protospacer adjacent motif (PAM).

25. The system of claim 21, wherein the Cas enzyme is Cas9, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17 Csx14, Csx10, Csx16, CsaX, Csx3, Csx1, Csx15, Csf1, Csf2, Csf3, Csf4, Cpf1, homologs thereof, orthologs thereof, or modified versions thereof.

26. The system of claim 21, wherein the Cas enzyme is Cas9.

27. The system of claim 21, wherein the Cas enzyme comprises one or more mutations.

28. The system of claim 21, wherein the Cas enzyme or a variant thereof cleaves the target sequence.

29. A system that targets a target sequence in a double-stranded DNA, the system comprising:
(i) a first DNA polynucleotide encoding a first RNA, the first RNA comprising:
(a) a first segment that hybridizes with a target sequence in a target strand of the double-stranded DNA; and
(b) a second segment that hybridizes with the first segment to form a double-stranded protein-binding motif;
(ii) a second DNA polynucleotide encoding a second RNA, wherein the second RNA hybridizes with a sequence in a non-target strand of the double-stranded DNA; and
(iii) a third DNA polynucleotide encoding a Cas enzyme or a variant thereof.

30. The system of claim 29, wherein the first DNA polynucleotide, the second DNA polynucleotide, and the third DNA polynucleotide are within a vector.

31. The system of claim 29, wherein the first DNA polynucleotide, the second DNA polynucleotide, and the third DNA polynucleotide are located on different vectors.

32. The system of claim 29, wherein the second RNA has about 14 to about 34 nucleotides.

33. The system of claim 29, wherein the target sequence is not immediately flanked by a protospacer adjacent motif (PAM).

34. The system of claim 29, wherein the target sequence is immediately flanked by a protospacer adjacent motif (PAM).

35. The system of claim 29, wherein the Cas enzyme is Cas9, Cas1, Cast1B, Cas2, Cas3, Cas4, Cas5, Cas6, Cas7, Cas8, Cas 10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, orthologs thereof, or modified versions thereof.

36. The system of claim 29, wherein the Cas enzyme is Cas9.

37. The system of claim 29, wherein the Cas enzyme or a variant thereof cleaves the target sequence.

38. A system that targets a target sequence in a double-stranded DNA, the system comprising:
(i) a first DNA polynucleotide encoding a first RNA, the first RNA comprising:
(a) a first segment that hybridizes with a target sequence in a target strand of the double-stranded DNA; and
(b) a second segment that hybridizes with the first segment to form a double-stranded protein-binding motif; and
(c) a third segment that hybridizes with a sequence in a non-target strand of the double-stranded DNA;
(ii) a second DNA polynucleotide encoding a Cas enzyme or a variant thereof.

39. The system of claim 38, wherein the first DNA polynucleotide and the second DNA polynucleotide are within a vector.

40. The system of claim 38, wherein the first DNA polynucleotide and the second DNA polynucleotide are located on different vectors.

41. The system of claim 38, wherein the third segment has about 14 to about 34 nucleotides.

42. The system of claim 38, wherein the target sequence is not immediately flanked by a protospacer adjacent motif (PAM).

43. The system of claim 38, wherein the target sequence is immediately flanked by a protospacer adjacent motif (PAM).

44. The system of claim 38, wherein the Cas enzyme is Cas9.

45. The system of claim 38, wherein the Cas enzyme or a variant thereof cleaves the target sequence.

46. A system that targets a target sequence in a double-stranded DNA, the system comprising:
(i) a first DNA polynucleotide encoding a first RNA, wherein the first RNA hybridizes with a target sequence in a target strand of a double-stranded DNA;
(ii) a second DNA polynucleotide encoding a second RNA, wherein the second RNA hybridizes with the first RNA to form a double-stranded protein-binding motif;
(iii) a third DNA polynucleotide encoding a third RNA, wherein the third RNA hybridizes with a sequence in a non-target strand of the double-stranded DNA; and
(iv) a fourth DNA polynucleotide encoding a Cas enzyme or a variant thereof.

47. The system of claim 46, wherein the first DNA polynucleotide, the second DNA polynucleotide, the third DNA polynucleotide, and the fourth DNA polynucleotide are within a vector.

48. The system of claim 46, wherein the first DNA polynucleotide, the second DNA polynucleotide, the third DNA polynucleotide, and the fourth DNA polynucleotide are located on different vectors.

49. The system of claim 46, wherein the third RNA has about 14 to about 34 nucleotides.

50. The system of claim 46, wherein the target sequence is not immediately flanked by a protospacer adjacent motif (PAM).

51. The system of claim 46, wherein the target sequence is immediately flanked by a protospacer adjacent motif (PAM).

52. The system of claim 46, wherein the Cas enzyme is Cas9.

53. The system of claim 46, wherein the Cas enzyme or a variant thereof cleaves the target sequence.

54. A kit comprising the system of any of claims 1-10 and 21-53.

55. A method of targeting a target sequence in a double-stranded DNA, the method comprising the step of contacting the double-stranded DNA with a system comprising:
(i) a first RNA, or a DNA polynucleotide encoding a first RNA, wherein the first RNA comprises:
(a) a first segment that hybridizes with a target sequence in a target strand of the double-stranded DNA;
(b) a second segment that hybridizes with the first segment to form a double-stranded protein-binding motif;
(ii) a second RNA, or a DNA polynucleotide encoding a second RNA, wherein the second RNA hybridizes with a sequence in a non-target strand of the double-stranded DNA; and
(iii) a Cas enzyme protein or a variant thereof, or a DNA polynucleotide or a RNA polynucleotide encoding a Cas enzyme or a variant thereof.

56. The method of claim 55, wherein the second RNA has about 14 to about 34 nucleotides.

57. The method of claim 55, wherein the target sequence is not immediately flanked by a protospacer adjacent motif (PAM).

58. The method of claim 55, wherein the target sequence is immediately flanked by a protospacer adjacent motif (PAM).

59. The method of claim 55, wherein the Cas enzyme is Cas9.

60. A method of targeting a target sequence in a double-stranded DNA, the method comprising the step of contacting the double-stranded DNA with a system comprising:
(i) a DNA-targeting RNA, or a DNA polynucleotide encoding a DNA-targeting RNA, wherein the DNA-targeting RNA comprises:
(a) a first segment that hybridizes with a target sequence in a target strand of the double-stranded DNA;
(b) a second segment that hybridizes with the first segment to form a double-stranded protein-binding motif; and
(c) a third segment that hybridizes with a sequence in a non-target strand of the double-stranded DNA; and
(ii) a Cas enzyme or a variant thereof, or a DNA polynucleotide or a RNA polynucleotide encoding a Cas enzyme or a variant thereof.

61. A method of targeting a target sequence in a double-stranded DNA in a cell, the method comprising the step of introducing into the cell a system comprising:
(i) a first RNA, or a DNA polynucleotide encoding a first RNA, wherein the first RNA comprises:
(a) a first segment that hybridizes with a target sequence in a target strand of the double-stranded DNA; and
(b) a second segment that hybridizes with the first segment to form a double-stranded protein-binding motif; and
(ii) a second RNA, or a DNA polynucleotide encoding a second RNA, wherein the second RNA hybridizes with a sequence in a non-target strand of the double-stranded DNA.

62. The method of claim 61, wherein the cell expresses a Cas enzyme.

63. The method of claim 61, further comprising delivering into the cell (i) a DNA polynucleotide encoding a Cas enzyme or a variant thereof; (ii) a RNA polynucleotide encoding a Cas enzyme or a variant thereof, or (iii) a Cas enzyme or a variant thereof.

64. A method of targeting a target sequence in a double-stranded DNA in a cell, the method comprising the step of introducing into the cell a DNA-targeting RNA, or a DNA polynucleotide encoding a DNA-targeting RNA, wherein the DNA-targeting RNA comprises:
(a) a first segment that hybridizes with a target sequence in a target strand of the double-stranded DNA;
(b) a second segment that hybridizes with the first segment to forth a double-stranded protein-binding motif; and
(c) a third segment that hybridizes with a sequence in a non-target strand of the double-stranded DNA.

65. The method of claim 64, wherein the cell expresses a Cas enzyme.

66. The method of claim 64, further comprising delivering into the cell (i) a DNA polynucleotide encoding a Cas enzyme or a variant thereof, (ii) a RNA polynucleotide encoding a Cas enzyme or a variant thereof, or (iii) a Cas enzyme or a variant thereof.


SEQUENCE LISTING (11 sequences)

SEQ ID NO: 1 - DNA, 2148 bp, Artificial Sequence
  2148-bp dsDNA substrate
cgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggcccaccaccaccaccaccacctggccaccacccatatgggatccgaattcgatatcttaattaagctgcaggagctcgtcgacgcggccgcactcgagtgagatctgactgaaaaaaaaaaaaaaaaaaaaaaaaaaaaaagtttaaacactagtccgctgagcaataactagcataaccccttggggcctctaaacgggtcttgaggggttttttgctgaaaggaggaactatatccgggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgaga

SEQ ID NO: 2 - DNA, 80 bp, Artificial Sequence
  80-bp ssDNA-1 substrate
aaagccacgtgtataagatacacctgcaaaggcggcacaaccccagtgccacgttgtgagttggatagttgtggaaagag

SEQ ID NO: 3 - DNA, 80 bp, Artificial Sequence
  80-bp ssDNA-2 substrate
ctctttccacaactatccaactcacaacgtggcactggggttgtgccgcctttgcaggtgtatcttatacacgtggcttt

SEQ ID NO: 4 - RNA, 36 nt, Artificial Sequence
  crRNA 580
agauacaccugcaaaggcggguuuuagagcuaugcu

SEQ ID NO: 5 - RNA, 36 nt, Artificial Sequence
  crRNA 591
ggcacugggguugugccgccguuuuagagcuaugcu

SEQ ID NO: 6 - DNA, 153 bp, Artificial Sequence
  DNA template for transcribing isgRNA 580
taatacgactcactataggagatacacctgcaaaggcgggttttagagctagaaatagcaagttaaaataaggctagtccgttatcaacttgaaaaagtggcaccgagtcggtgcttttttttttacacggcggaaacgtccacatagaatat

SEQ ID NO: 7 - DNA, 151 bp, Artificial Sequence
  DNA template for transcribing isgRNA 591
taatacgactcactataggcactggggttgtgccgccgttttagagctagaaatagcaagttaaaataaggctagtccgttatcaacttgaaaaagtggcaccgagtcggtgcttttttttttgtttccgccgtgttggggtcacggtgca

SEQ ID NO: 8 - RNA, 28 nt, Artificial Sequence
  iRNA 580
ugugccgccuuugcagguguaucuuaua

SEQ ID NO: 9 - RNA, 28 nt, Artificial Sequence
  iRNA 591
caaaggcggcacaaccccagugccacgu

SEQ ID NO: 10 - DNA, 118 bp, Artificial Sequence
  DNA template for transcribing  sgRNA 580
taatacgactcactataggagatacacctgcaaaggcgggttttagagctagaaatagcaagttaaaataaggctagtccgttatcaacttgaaaaagtggcaccgagtcggtgcttt

SEQ ID NO: 11 - DNA, 116 bp, Artificial Sequence
  DNA template for transcribing sgRNA 591
taatacgactcactataggcactggggttgtgccgccgttttagagctagaaatagcaagttaaaataaggctagtccgttatcaacttgaaaaagtggcaccgagtcggtgcttt